(19) United States
(12) Patent Application Publication
(10) Pub. No.: US 2002/0015735 A1
(43) Pub. Date: Feb. 7, 2002

(54) Sustained-release formulation of a cyclooxygenase-2 inhibitor

(76) Inventors: David B. Hedden, Ann Arbor, MI (US); Sreekant Nadkarni, Gurnee, IL (US)

Correspondence Address: Pharmacia Corporation, Patent Department Central, 1820, P.O. Box 5110, Chicago, IL, 60680-5110, US

(21) Appl. No.: 09/742,906

(22) Filed: Dec. 20, 2000

Related U.S. Application Data
(60) Provisional application No. 60/171,738, filed on Dec. 22, 1999.
(60) Provisional application No. 60/181,635, filed on Feb. 10, 2000.
(60) Provisional application No. 60/202,269, filed on May 5, 2000.

(51) Int. Cl. A61K 9/14; A61K 31/44; A61K 31/415; A61K 31/135

(52) U.S. Cl. 424/488; 424/487; 514/406; 514/349; 514/352; 514/646

(57) ABSTRACT

There is provided an orally deliverable pharmaceutical composition comprising a selective cyclooxygenase-2 inhibitory drug of low water solubility such as celecoxib and a release-extending polymer. The composition is useful in treatment of cyclooxygenase-2 mediated conditions and disorders by once-a-day administration.

This application claims priority of U.S. provisional application Ser. No. 60/171,738 filed on Dec. 22, 1999, U.S. provisional application Ser. No. 60/181,635 filed on Feb. 10, 2000, and U.S. provisional application Ser. No. 60/202,269 filed on May 5, 2000.

### FIELD OF THE INVENTION

This invention relates to orally deliverable pharmaceutical compositions containing a selective cyclooxygenase-2 (COX-2) inhibitory drug as an active ingredient, to processes for preparing such compositions, to methods of treatment of COX-2 mediated disorders comprising orally administering such compositions to a subject, and to use of such compositions in manufacture of medicaments.

### BACKGROUND OF THE INVENTION

Numerous compounds have been reported having therapeutically and/or prophylactically useful selective COX-2 inhibitory effect, and having utility in treatment or prevention of specific COX-2 mediated disorders or of such disorders in general. Among such compounds are a large number of substituted pyrazolyl benzenesulfonamides as reported in U.S. Pat. No. 5,760,068 to Talley et al., including for example the compound 4-[5-(4-methylphenyl)-3-(trifluoromethyl)-1H-pyrazol-1-yl]benzenesulfonamide, also referred to herein as celecoxib (I), and the compound 4-[5-(3-fluoro-4-methox yphenyl)-3-difluoromethyl)-1H-pyrazol-1-yl]benzenesulfonamide, also referred to herein as deracoxib (II).

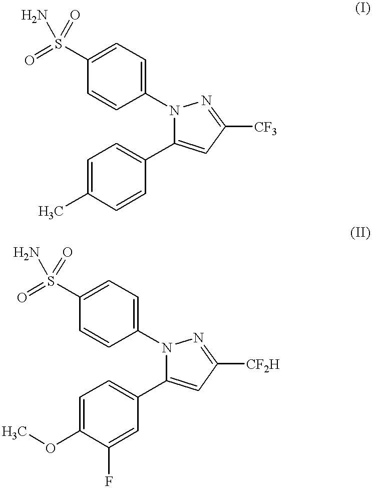

Other compounds reported to have therapeutically and/or prophylactically useful selective COX-2 inhibitory effect are substituted isoxazolyl benzenesulfonamides as reported in U.S. Pat. No. 5,633,272 to Talley et al., including the compound 4-[5-methyl-3-phenylisoxazol-4-yl]benzenesulfonamide, also referred to herein as valdecoxib (III).

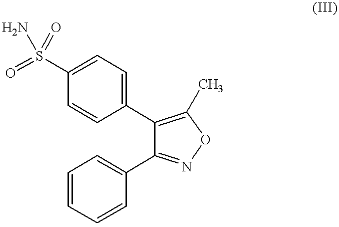

Still other compounds reported to have therapeutically and/or prophylactically useful selective COX-2 inhibitory effect are substituted (methylsulfonyl)phenyl furanones as reported in U.S. Pat. No. 5,474,995 to Ducharme et al, including the compound 3-phenyl-4-[4-(methylsulfonyl)phenyl]-5H-furan-2-one, also referred to herein as rofecoxib (IV).

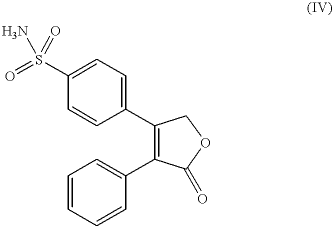

U.S. Pat. No. 5,981,576 to Belley et al. discloses a further series of (methylsulfonyl)phenyl furanones said to be useful as selective COX-2 inhibitory drugs, including 3-(1-cyclopropylmethoxy)-5,5-dimethyl-4-[4-(methylsulfonyl)phenyl]-5H-furan-2-one and 3-(1-cyclopropylethoxy)-5,5-dimethyl-4-[4-(methylsulfonyl)phenyl]-5H-furan-2-one.

U.S. Pat. No. 5,861,419 to Dube et al. discloses substituted pyridines said to be useful as selective COX-2 inhibitory drugs, including for example the compound 5-chloro-3-(4-methylsulfonyl)phenyl-2-(2-methyl-5-pyridinyl)pyridine (V).

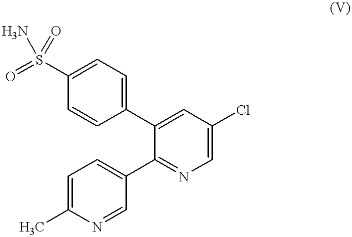

European Patent Application No. 0 863 134 discloses the compound 2-(3,5-difluorophenyl)-3-[4-(methylsulfonyl)phenyl]-2-cyclopenten-1-one said to be useful as a selective COX-2 inhibitory drug.

U.S. Pat. No. 6,034,256 discloses a series of benzopyrans said to be useful as selective COX-2 inhibitory drugs, including the compound (S)-6,8-dichloro-2-(trifluoromethyl)-2H-1-benzopyran-3-carboxylic acid (VI).

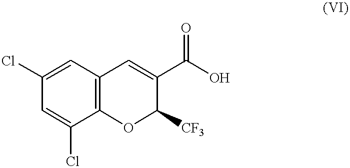

A need for formulated compositions of selective COX-2 inhibitory drugs, in particular sustained-release compositions, exists. Sustained-release drug-delivery systems can provide many benefits over conventional dosage forms. Generally, sustained-release preparations provide a longer period of therapeutic or prophylactic response compared to conventional rapid release dosage forms. For example, in treatment of pain, sustained-release formulations are useful to maintain relatively constant analgesic drug release rates over a period of time, for example 12-24 hours, so that blood serum concentration of the drug remains at a therapeutically effective level for a longer duration than is possible with a conventional dosage form of the drug. In addition, whereas standard dosage forms typically exhibit high initial drug release rates that can result in unnecessarily elevated blood serum levels of the drug, sustained-release formulations can help maintain blood serum levels of the drug at or slightly above the therapeutically effective threshold. Such reduced fluctuation in blood serum concentration of the drug can also help prevent excess dosing.

Furthermore, sustained-release compositions, by optimizing the kinetics of delivery, also increase patient compliance as patients are less likely to miss a dose with less frequent administration, particularly when a once-a-day dosage regimen is possible; less frequent administration also increases patient convenience. Additionally, sustained-release formulations can reduce overall healthcare costs.

Although the initial cost of sustained-release delivery systems may be greater than the costs associated with conventional delivery systems, average costs of extended treatment over time can be lower due to less frequent dosing, enhanced therapeutic benefit, reduced side-effects, and a reduction in the time required to dispense and administer the drug and monitor patient compliance.

Many selective COX-2 inhibitory compounds, in particular those having low solubility in water, including celecoxib, deracoxib, valdecoxib and rofecoxib, possess physical and chemical properties which make them poorly amenable to sustained-release dosage formulation. These physical and chemical properties have presented practical difficulties in formulating longer-acting low solubility selective COX-2 inhibitory drugs for oral administration.

Illustratively, the formulation of celecoxib for effective sustained-release oral administration to a subject has hitherto been complicated by the unique physical, chemical and pharmacological properties of celecoxib, particularly its exceptionally low solubility in aqueous media, its relatively high dose requirement, and patient-to-patient variability in its absorption. Drugs with extremely high or low aqueous solubility are known to be difficult to incorporate into effective sustained-release delivery systems (Lieberman et al., ed. (1990) Pharmaceutical Dosage Forms: Tablets, 2nd ed., Vol. 3. Marcel Dekker, Inc., New York.) For example, a lower solubility limit for sustained-release products has been reported to be about 0.1 mg/ml (Fincher (1968) “Particle size of drugs and its relationship to absorption and activity”, J. Pharma. Sci., 57, 1825), whereas celecoxib has a solubility of 5 μg/ml. Drugs having a relatively high oral dose requirement are also known to be poor candidates for sustained-release systems, in part because inclusion of a sufficient dose to provide prolonged therapeutic effect and of the release-sustaining mechanism tend to result in an unacceptably large volume of product (Lieberman et al., op. cit., p. 206). Finally, drugs that are absorbed at a rate that varies significantly among treated subjects have also been considered inferior candidates for sustained-release systems, in part because such systems normally target a blood concentration of the drug not greatly in excess of the threshold concentration for therapeutic effectiveness, and subjects showing relatively poor absorption can fail to reach that threshold concentration (Lieberman et al., op. cit., p. 207).

For these and other reasons, therefore, it would be a difficult but much desired advance in the art to provide an effective sustained-release formulation of a selective COX-2 inhibitory drug of low solubility, such as celecoxib.

A wide variety of controlled-release, slow-release, programmed-release, timed-release, pulse-release, sustained-release or extended-release technologies are known in the art for drugs other than those addressed in the present invention. Typically such technologies involve formulating the drug in a polymer matrix from which the drug is gradually released, or protecting the drug from immediate release by means of a barrier layer which degrades over time in the gastrointestinal tract. Examples of barrier layers include liposomes, nanocapsules, microcapsules and coatings on granules, beads or tablets. Dosage forms can be liquids (e.g., suspensions) or unit dose articles (e.g., tablets, capsules, soft capsules).

Illustrative processes that have been contemplated for preparing controlled-release, slow-release, programmed-release, timed-release, pulse-release, sustained-release or extended-release formulations of opioids, NSAIDs and other analgesic, antipyretic and anti-inflammatory drugs are disclosed in the patents and publications listed below, each of which is individually incorporated herein by reference.

U.S. Pat. No. 3,362,880 to Jeffries.

U.S. Pat. No. 4,308,251 to Dunn & Lampard.

U.S. Pat. No. 4,316,884 to Alam & Eichel.

U.S. Pat. No. 4,571,333 to Hsias & Kent.

U.S. Pat. No. 4,601,894 to Hanna & Vadino.

U.S. Pat. No. 4,708,861 to Popescu et al.

U.S. Pat. No. 4,749,575 to Rotman.

U.S. Pat. No. 4,765,989 to Wong et al.

U.S. Pat. No. 4,795,641 to Kashdan.

U.S. Pat. No. 4,803,079 to Hsias & Kent.

U.S. Pat. No. 4,847,093 to Ayer & Wong.

U.S. Pat. No. 4,867,985 to Heafield et al

U.S. Pat. No. 4,892,778 to Theeuwes et al.

U.S. Pat. No. 4,940,588 to Sparks & Geoghegan.

U.S. Pat. No. 4,975,284 to Stead & Nabahi.

U.S. Pat. No. 4,980,175 to Chavkin & Mackles.

U.S. Pat. No. 5,055,306 to Barry et al.

U.S. Pat. No. 5,082,668 to Wong et al

U.S. Pat. No. 5,160,742 to Mazer et al.

U.S. Pat. No. 5,160,744 to Jao et al.

U.S. Pat. No. 5,190,765 to Jao et al.

U.S. Pat. No. 5,273,760 to Oshlack et al.

U.S. Pat. No. 5,275,820 to Chang.

U.S. Pat. No. 5,292,534 to Valentine & Valentine.

U.S. Pat. No. 5,296,236 to Santus & Golzi.

U.S. Pat. No. 5,415,871 to Pankhania et al

U.S. Pat. No. 5,427,799 to Valentine & Valentine.

U.S. Pat. No. 5,451,409 to Rencher et al.

U.S. Pat. No. 5,455,046 to Baichwal.

U.S. Pat. No. 5,460,825 to Roche.

U.S. Pat. No. 5,472,711 to Baichwal.

U.S. Pat. No. 5,472,712 to Oshlack et al.

U.S. Pat. No. 5,478,574 to Mendell.

U.S. Pat. No. 5,518,730 to Fuisz.

U.S. Pat. No. 5,523,095 to Modi.

U.S. Pat. No. 5,527,545 to Santus et al.

U.S. Pat. No. 5,536,505 to Wilson et al.

U.S. Pat. No. 5,571,533 to Santus et al.

U.S. Pat. No. 5,674,533 to Santus et al.

U.S. Pat. No. 5,773,025 to Baichwal.

U.S. Pat. No. 5,858,344 to Muller & Cremer.

U.S. Pat. No. 6,093,420 to Baichwal.

International Patent Publication No. WO 87/00044.

International Patent Publication No. WO 89/08119.

International Patent Publication No. WO 91/16920.

International Patent Publication No. WO 92/13547.

International Patent Publication No. WO 93/10760.

International Patent Publication No. WO 93/10769.

International Patent Publication No. WO 93/12765.

International Patent Publication No. WO 93/17673.

International Patent Publication No. WO 95/14460.

International Patent Publication No. WO 96/16638.

International Patent Publication No. WO 98/01117.

International Patent Publication No. WO 99/12524.

International Patent Publication No. WO 99/51209.

International Patent Publication No. WO 99/61005.

International Patent Publication No. WO 00/18374.

International Patent Publication No. WO 00/33818.

International Patent Publication No. WO 00/40205.

Belgian Pat ent Application No. 9 00 824.

European Patent Application No. 0 147 780.

European Patent Application No. 0 438 249.

European Patent Application No. 0 516 141.

European Patent Application No. 0 875 245.

European Patent Application No. 0 945 137.

French Patent Application No. 2 58 4 604.

Japanese Laid-Open Patent Applicati on No. 56/030402.

Japanese Laid-Open Patent Application No. 60/072813.

Japanese Laid-Open Patent Application No. 63/174925.

Japanese Laid-Open Patent Application No. 10/298064.

Several factors influence dissolution in a solvent medium of a drug from its carrier, including the surface area of the drug presented to the solvent medium, the solubility of the drug in the solvent medium, and the driving forces of the saturation concentration of dissolved materials in the solvent medium. Notwithstanding these factors, a strong correlation has been established between the in vitro dissolution time determined for a dosage form and the in vivo drug release rate. This correlation is so firmly established in the art that dissolution time has become generally descriptive of drug release potential for the active component of the particular unit dosage composition. In view of this relationship, it is clear that dissolution time determined for a composition is one of the important fundamental characteristics for consideration when evaluating sustained-release compositions.

### SUMMARY OF THE INVENTION

According to the present invention, a composition is provided wherein a poorly water-soluble selective COX-2 inhibitory drug exhibits a sustained-release profile. In one embodiment, the composition comprises a therapeutically effective amount of such a drug, one or more pharmaceutically acceptable polymers and, optionally, one or more pharmaceutically acceptable excipients other than such polymers. In this embodiment, the composition provides an in vitro dissolution profile, following placement in a standard dissolution medium, exhibiting (a) release of about 5% to about 35% of the drug 2 hours after such placement; (b) release of about 10% to about 85% of the drug 8 hours after such placement; and (c) release of about 30% to about 90% of the drug 18 hours after such placement.

Polymers useful in the invention are, in one embodiment, swellable or erodible polymers and, more preferably, release-extending swellable or erodible polymers. A swellable polymer is a polymer that, when placed in an aqueous medium, absorbs water and swells, forming a matrix. An erodible polymer is defined herein as a polymer that, when present as a matrix or coating in or on a tablet or bead comprising a drug, and where the tablet or bead is placed in an aqueous medium, progressively from the outside of the tablet or bead inward to the center thereof, dissolves or disperses in the medium. A release-extending swellable or erodible polymer is defined herein as a polymer that, when present in a formulated composition of a drug, causes the drug to be released to an aqueous medium at a slower rate than in the absence of such polymer.

In another embodiment, a polymer useful in the invention is neither highly swellable nor erodible as defined above, but, when present as a coating on a tablet or bead comprising a drug, has release-extending properties. Such a polymer is preferably used in combination with a water-soluble polymer such that when the coated tablet or bead is placed in an aqueous medium the coating becomes porous and permits slow release of the drug.

In a further embodiment the composition comprises a therapeutically effective amount of a poorly water-soluble selective COX-2 inhibitory drug, a substantial portion or all of which is distributed in a matrix comprising one or more pharmaceutically acceptable swellable polymers. In this embodiment the swellable polymers comprise hydroxypropylmethylcellulose (HPMC) having a viscosity, 2% in water, of about 100 to about 8,000 cP. Optionally the composition further comprises one or more pharmaceutically acceptable excipients other than such polymers.

In a still further embodiment the composition comprises a multiplicity of solid beads comprising a therapeutically effective amount of a poorly water-soluble selective COX-2 inhibitory drug. A substantial portion or all of the beads further comprise one or more release-extending polymers forming a coating on the beads. Preferably the release-extending polymers forming the coating comprise ethylcellulose or a polymer or copolymer of acrylic and/or methacrylic acids or esters thereof.

Surprisingly, compositions of the invention provide, by oral administration thereof, therapeutically effective sustained-release delivery of selective COX-2 inhibitory drugs such as celecoxib, in spite of the particular difficulties alluded to above, including low solubility, high dose requirement and patient-to-patient variability in absorption rate. The inventors have also had to overcome problems associated with low compressibility of celecoxib as well as its other physical and chemical properties. Preferred sustained-release celecoxib formulations of the invention have been found to possess improved bioavailability, chemical stability, physical stability, dissolution profiles, safety, and/or other improved pharmacokinetic, chemical, biological and/or physical properties.

The present invention comprises pharmaceutical compositions, unit dosage forms based thereon, and methods for the preparation and use of both. Other features of this invention will be in part apparent and in part pointed out hereinafter.

### BRIEF DESCRIPTION OF THE DRAWINGS

FIG. 1 shows the in vitro dissolution profiles of eight formulations M4 to M11 wherein celecoxib is distributed in a HPMC matrix. The composition of each formulation is shown in Table 3 herein.

FIG. 2 shows the in vitro dissolution profiles of eight formulations M12 to M21 wherein celecoxib is distributed in a HPMC matrix. The composition of each formulation is shown in Table 4 herein.

FIG. 3 shows the in vitro dissolution profiles of eight formulations S1 to S8 wherein celecoxib is present in beads having a polymer coating. The composition of each formulation is shown in Table 7 herein.

FIG. 4 shows the in vitro dissolution profiles of four formulations Q5 to Q8 wherein valdecoxib is distributed in a HPMC matrix. The composition of each formulation is shown in Table 9 herein.

FIG. 5 shows the in vitro dissolution profiles of six formulations Q11 to Q16 wherein valdecoxib is distributed in a HPMC matrix. The composition of each formulation is shown in Table 10 herein.

FIG. 6 shows in vivo pharmacokinetic parameters of three formulations M12, M13 and M17 wherein celecoxib is distributed in a HPMC matrix, and formulation S4 wherein celecoxib is present in beads having a polymer coating, by comparison with an immediate release tablet formulation. The compositions of these formulations are shown in Tables 4 and 7 herein.

FIG. 7 shows in vivo pharmacokinetic parameters of three formulations Q17, Q18, and Q20 wherein valdecoxib is distributed in a HPMC matrix, by comparison with an immediate release tablet formulation for comparison. The compositions of these formulations are shown in Table 11 herein.

### DETAILED DESCRIPTION OF THE INVENTION

Selective COX-2 inhibitory drugs for which the present invention is useful are drugs that inhibit COX-2 to a therapeutically useful degree while causing markedly less inhibition of cyclooxygenase-1 (COX-1) than conventional nonsteroidal anti-inflammatory drugs (NSAIDs).

The invention applies particularly to selective COX-2 inhibitory drugs of low water solubility, especially those having a solubility in distilled water at 25° C. lower than about 10 g/l, preferably lower than about 1 g/l, and most preferably lower than about 0.1 g/l.

The poorly water-soluble selective COX-2 inhibitory drug can be any such drug known in the art, including without limitation compounds disclosed in the patents and publications listed below, each of which is individually incorporated herein by reference.

U.S. Pat. No. 5,344,991 to Reitz & Li.

U.S. Pat. No. 5,380,738 to Norman et al.

U.S. Pat. No. 5,393,790 to Reitz et al.

U.S. Pat. No. 5,401,765 to Lee.

U.S. Pat. No. 5,418,254 to Huang & Reitz.

U.S. Pat. No. 5,420,343 to Koszyk & Weier.

U.S. Pat. No. 5,434,178 to Talley & Rogier.

U.S. Pat. No. 5,436,265 to Black et al.

Above-cited U.S. Pat. No. 5,466,823.

U.S. Pat. No. 5,474,995 to Ducharme et al

U.S. Pat. No. 5,475,018 to Lee & Bertenshaw.

U.S. Pat. No. 5,486,534 to Lee et al.

U.S. Pat. No. 5,510,368 to Lau et al.

U.S. Pat. No. 5,521,213 to Prasit et al.

U.S. Pat. No. 5,536,752 to Ducharme et al.

U.S. Pat. No. 5,543,297 to Cromlish et al.

U.S. Pat. No. 5,547,975 to Talley et al.

U.S. Pat. No. 5,550,142 to Ducharme et al.

U.S. Pat. No. 5,552,422 to Gauthier et aL

U.S. Pat. No. 5,585,504 to Desmond et al.

U.S. Pat. No. 5,593,992 to Adams et aL

U.S. Pat. No. 5,596,008 to Lee.

U.S. Pat. No. 5,604,253 to Lau et al.

U.S. Pat. No. 5,604,260 to Guay & Li.

U.S. Pat. No. 5,616,458 to Lipsky et al.

U.S. Pat. No. 5,616,601 to Khanna et al.

U.S. Pat. No. 5,620,999 to Weier et al.

Above-cited U.S. Pat. No. 5,633,272.

U.S. Pat. No. 5,639,780 to Lau et al

U.S. Pat. No. 5,643,933 to Talley et al.

U.S. Pat. No. 5,658,903 to Adams et aL

U.S. Pat. No. 5,668,161 to Talley et al

U.S. Pat. No. 5,670,510 to Huang & Reitz.

U.S. Pat. No. 5,677,318 to Lau.

U.S. Pat. No. 5,681,842 to Dellaria & Gane.

U.S. Pat. No. 5,686,460 to Nicolai et al.

U.S. Pat. No. 5,686,470 to Weier et al.

U.S. Pat. No. 5,696,143 to Talley et al.

U.S. Pat. No. 5,710,140 to Ducharme et aL

U.S. Pat. No. 5,716,955 to Adams et al.

U.S. Pat. No. 5,723,485 to Gungor & Teulon.

U.S. Pat. No. 5,739,166 to Reitz et al.

U.S. Pat. No. 5,741,798 to Lazer et al.

U.S. Pat. No. 5,756,499 to Adams et al.

U.S. Pat. No. 5,756,529 to Isakson & Talley.

U.S. Pat. No. 5,776,967 to Kreft et al.

U.S. Pat. No. 5,783,597 to Beers & Wachter.

U.S. Pat. No. 5,789,413 to Black et al.

U.S. Pat. No. 5,807,873 to Nicolai & Teulon.

U.S. Pat. No. 5,817,700 to Dube et al.

U.S. Pat. No. 5,830,911 to Failli et al

U.S. Pat. No. 5,849,943 to Atkinson & Wang.

U.S. Pat. No. 5,859,036 to Sartori et al.

U.S. Pat. No. 5,861,419 to Dube et al

U.S. Pat. No. 5,866,596 to Sartori & Teulon.

U.S. Pat. No. 5,869,524 to Failli.

U.S. Pat. No. 5,869,660 to Adams et al.

U.S. Pat. No. 5,883,267 to Rossen et al.

U.S. Pat. No. 5,892,053 to Zhi et al.

U.S. Pat. No. 5,922,742 to Black et al.

U.S. Pat. No. 5,929,076 to Adams & Garigipati.

U.S. Pat. No. 5,932,598 to Talley et al.

U.S. Pat. No. 5,935,990 to Khanna et al.

U.S. Pat. No. 5,945,539 to Haruta et al

U.S. Pat. No. 5,958,978 to Yamazaki et al.

U.S. Pat. No. 5,968,958 to Guay et al.

U.S. Pat. No. 5,972,950 to Nicolai & Teulon.

U.S. Pat. No. 5,973,191 to Mamett & Kalgutkar.

U.S. Pat. No. 5,981,576 to Belley et al

U.S. Pat. No. 5,994,381 to Haruta et al.

U.S. Pat. No. 6,002,014 to Haruta et al.

U.S. Pat. No. 6,004,960 to Li et al

U.S. Pat. No. 6,005,000 to Hopper et al.

U.S. Pat. No. 6,020,343 to Belley et al.

U.S. Pat. No. 6,020,347 to DeLaszlo & Hagmann.

U.S. Pat. No. 6,034,256 to Carter et al.

U.S. Pat. No. 6,040,319 to Corley et al.

U.S. Pat. No. 6,040,450 to Davies et al.

U.S. Pat. No. 6,046,208 to Adams et al.

U.S. Pat. No. 6,046,217 to Friesen et al.

U.S. Pat. No. 6,057,319 to Black et al.

U.S. Pat. No. 6,063,804 to De Nanteuil et al.

U.S. Pat. No. 6,063,807 to Chabrier de Lassauniere & Broquet.

U.S. Pat. No. 6,071,954 to LeBlanc et al.

U.S. Pat. No. 6,077,868 to Cook et al.

U.S. Pat. No. 6,077,869 to Sui & Wachter.

U.S. Pat. No. 6,083,969 to Ferro et al.

U.S. Pat. No. 6,096,753 to Spohr et al.

U.S. Pat. No. 6,133,292 to Wang et al.

International Patent Publication No. WO 94/15932.

International Patent Publication No. WO 96/19469.

International Patent Publication No. WO 96/26921.

International Patent Publication No. WO 96/31509.

International Patent Publication No. WO 96/36623.

International Patent Publication No. WO 96/38418.

International Patent Publication No. WO 97/03953.

International Patent Publication No. WO 97/10840.

International Patent Publication No. WO 97/13755.

International Patent Publication No. WO 97/13767.

International Patent Publication No. WO 97/25048.

International Patent Publication No. WO 97/30030.

International Patent Publication No. WO 97/34882.

International Patent Publication No. WO 97/46524.

International Patent Publication No. WO 98/04527.

International Patent Publication No. WO 98/06708.

International Patent Publication No. WO 98/07425.

International Patent Publication No. WO 98/17292.

International Patent Publication No. WO 98/21195.

International Patent Publication No. WO 98/22457.

International Patent Publication No. WO 98/32732.

International Patent Publication No. WO 98/41516.

International Patent Publication No. WO 98/43966.

International Patent Publication No. WO 98/45294.

International Patent Publication No. WO 98/47871.

International Patent Publication No. WO 99/01130.

International Patent Publication No. WO 99/01131.

International Patent Publication No. WO 99/01452.

International Patent Publication No. WO 99/01455.

International Patent Publication No. WO 99/10331.

International Patent Publication No. WO 99/10332.

International Patent Publication No. WO 99/11605.

International Patent Publication No. WO 99/12930.

International Patent Publication No. WO 99/14195.

International Patent Publication No. WO 99/14205.

International Patent Publication No. WO 99/15505.

International Patent Publication No. WO 99/23087.

International Patent Publication No. WO 99/24404.

International Patent Publication No. WO 99/25695.

International Patent Publication No. WO 99/35130.

International Patent Publication No. WO 99/61016.

International Patent Publication No. WO 99/61436.

International Patent Publication No. WO 99/62884.

International Patent Publication No. WO 99/64415.

International Patent Publication No. WO 00/01380.

International Patent Publication No. WO 00/08024.

International Patent Publication No. WO 00/10993.

International Patent Publication No. WO 00/13684.

International Patent Publication No. WO 00/18741.

International Patent Publication No. WO 00/18753.

International Patent Publication No. WO 00/23426.

International Patent Publication No. WO 00/24719.

International Patent Publication No. WO 00/26216.

International Patent Publication No. WO 00/31072.

International Patent Publication No. WO 00/40087.

International Patent Publication No. WO 00/56348.

European Patent Application No. 0 799 823.

European Patent Application No. 0 846 689.

European Patent Application No. 0 863 134.

European Patent Application No. 0 985 666.

Compositions of the invention are especially useful for compounds having the formula (VI):

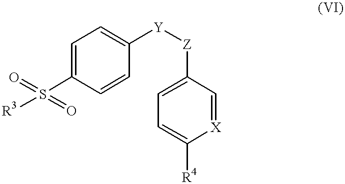

where R³ is a methyl or amino group, R⁴ is hydrogen or a C₁₋₄ alkyl or alkoxy group, X is N or CR⁵ where R⁵ is hydrogen or halogen, and Y and Z are independently carbon or nitrogen atoms defining adjacent atoms of a five- to six-membered ring that is unsubstituted or substituted at one or more positions with oxo, halo, methyl or halomethyl groups. Preferred such five- to six-membered rings are cyclopentenone, furanone, methylpyrazole, isoxazole and pyridine rings substituted at no more than one position.

Illustratively, compositions of the invention are suitable for celecoxib, deracoxib, valdecoxib, rofecoxib, 5-chloro-3-(4-methylsulfonyl)phenyl-2-(2-methyl-5-pyridinyl)pyridine, 2-(3,5-difluorophenyl)-3-[4-(methylsulfonyl)phenyl]-2-cyclopenten-1-one and (S)-6,8-dichloro-2-(trifluoromethyl)-2H-1-benzopyran-3-carboxylic acid, more particularly celecoxib and valdecoxib, and most particularly celecoxib.

The present invention provides sustained-release pharmaceutical compositions and dosage forms suitable for oral administration, comprising a selective COX-2 inhibitory drug of low solubility in water. Where the invention is illustrated herein with particular reference to celecoxib or valdecoxib, it will be understood that any other selective COX-2 inhibitory drug of low solubility in water can, if desired, be substituted in whole or in part for celecoxib or valdecoxib in compositions herein described.

Compositions of the invention comprise one or more orally deliverable dose units. Each dose unit comprises a selective COX-2 inhibitory drug, illustratively celecoxib, in a therapeutically effective amount that is preferably about 5 mg to about 1000 mg, more preferably about 10 mg to about 1000 mg.

It will be understood that a therapeutically effective amount of a selective COX-2 inhibitory drug for a subject is dependent inter alia on the body weight of the subject. Where the drug is celecoxib and the subject is a child or a small animal (e.g., a dog), for example, an amount of celecoxib relatively low in the preferred range of about 10 mg to about 1000 mg is likely to provide blood serum concentrations consistent with therapeutic effectiveness. Where the subject is an adult human or a large animal (e.g., a horse), achievement of such blood serum concentrations of celecoxib are likely to require dose units containing a relatively greater amount of celecoxib. For an adult human, a therapeutically effective amount of celecoxib per dose unit in a composition of the present invention is typically about 50 mg to about 400 mg. Especially preferred amounts of celecoxib per dose unit are about 100 mg to about 200 mg, for example about 100 mg or about 200 mg.

For other selective COX-2 inhibitory drugs, an amount of the drug per dose unit can be in a range known to be therapeutically effective for such drugs.

Preferably, the amount per dose unit is in a range providing therapeutic equivalence to celecoxib in the dose ranges indicated immediately above. Celecoxib compositions of the invention exhibit improved performance as selective COX-2 inhibitory medications. In particular, these compositions provide celecoxib to a subject at a dosage and release rate sufficient to provide prolonged inhibition of COX-2 and thus confer the desired therapeutic benefit for an extended period, typically up to 24 hours, yet maintain a safe clearance time for celecoxib. Three primary mechanisms by which drugs are removed from the body include hepatic metabolism, renal excretion, and elimination of the drug into bile with subsequent excretion. The phrase “clearance time” as used herein refers to the time taken for the sum of all clearance processes to eliminate the drug from the body.

Oral administration of a sustained-release celecoxib composition of the invention results in reduced early blood plasma celecoxib concentrations compared with previously disclosed celecoxib compositions administered at equal dose. In more general terms, sustained-release compositions of the invention achieve a therapeutic threshold of plasma drug concentration without providing excessive or unnecessarily high plasma drug concentrations at early time points following administration. However, the particular therapeutic threshold associated with a given drug depends on the individual subject and on the therapeutic indication for which the drug is being used. Illustratively, a therapeutic threshold for celecoxib concentration in plasma is about 50 ng/ml to about 200 ng/ml, for example about 100 ng/ml.

Celecoxib used in the process and compositions of the present invention can be prepared by a process known per se, for example by processes set forth in U.S. Pat. No. 5,466,823 to Talley et al. or in U.S. Pat. No. 5,892,053 to Zhi & Newaz, both incorporated herein by reference. Other selective COX-2 inhibitory drugs can be prepared by processes known per se, including processes set forth in patent publications disclosing such drugs; for example in the case of valdecoxib in above-cited U.S. Pat. No. 5,633,272, and in the case of rofecoxib in above-cited U.S. Pat. No. 5,474,995.

Celecoxib compositions of the present invention comprise celecoxib in a daily dosage amount of about 10 mg to about 1000 mg. Preferably, such compositions comprise celecoxib in a daily dosage amount of about 50 mg to about 800 mg, more preferably about 75 mg to about 400 mg, and still more preferably about 100 mg to about 200 mg.

Compositions of the present invention are preferably in the form of discrete solid unit dose articles such as capsules or tablets. Preferably, a single such article or a small plurality (up to about 10, more preferably no more than about 4) of such articles is sufficient to provide the daily dose. Thus an embodiment of the invention is a composition as described herein above comprising one or more discrete solid orally deliverable unit dose articles, for example capsules or tablets, each comprising celecoxib.

Such unit dose articles typically contain about 10 mg to about 400 mg of celecoxib, for example, a 10, 20, 37.5, 50, 75, 100, 125, 150, 175, 200, 250, 300, 350 or 400 mg dose of celecoxib. Preferred articles are tablets or capsules containing about 25 mg to about 400 mg, more preferably about 50 mg to about 200 mg, of celecoxib. A particular unit dosage form can be selected to accommodate the desired frequency of administration used to achieve a specified daily dosage.

A composition of the invention preferably contains about 1% to about 95%, preferably about 10% to about 90%, more preferably about 25% to about 85%, and still more preferably about 30% to about 80%, by weight of the selective COX-2 inhibitory drug, alone or in intimate mixture with one or more excipients.

Compositions of the invention are useful in treatment and prevention of a very wide range of disorders mediated by COX-2, including but not restricted to disorders characterized by inflammation, pain and/or fever. Such compositions are especially useful as anti-inflammatory agents, such as in treatment of arthritis, with the additional benefit of having significantly less harmful side effects than compositions of conventional nonsteroidal anti-inflammatory drugs (NSAIDs) that lack selectivity for COX-2 over COX-1. In particular, compositions of the invention have reduced potential for gastrointestinal toxicity and gastrointestinal irritation including upper gastrointestinal ulceration and bleeding, reduced potential for renal side effects such as reduction in renal function leading to fluid retention and exacerbation of hypertension, reduced effect on bleeding times including inhibition of platelet function, and possibly a lessened ability to induce asthma attacks in aspirin-sensitive asthmatic subjects, by comparison with compositions of conventional NSAIDs. Thus compositions of the invention are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinemia, hemophilia or other bleeding problems; kidney disease; or in patients prior to surgery or patients taking anticoagulants.

Contemplated compositions are useful to treat a variety of arthritic disorders, including but not limited to rheumatoid arthritis, spondyloarthropathies, gouty arthritis, osteoarthritis, systemic lupus erythematosus and juvenile arthritis.

Such compositions are useful in treatment of asthma, bronchitis, menstrual cramps, preterm labor, tendinitis, bursitis, allergic neuritis, cytomegalovirus infectivity, apoptosis including HIV-induced apoptosis, lumbago, liver disease including hepatitis, skin-related conditions such as psoriasis, eczema, acne, bums, dermatitis and ultraviolet radiation damage including sunburn, and post-operative inflammation including that following ophthalmic surgery such as cataract surgery or refractive surgery.

Such compositions are useful to treat gastrointestinal conditions such as inflammatory bowel disease, Crohn's disease, gastritis, irritable bowel syndrome and ulcerative colitis.

Such compositions are useful in treating inflammation in such diseases as migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such compositions are useful in treatment of ophthalmic diseases, such as retinitis, conjunctivitis, retinopathies, uveitis, ocular photophobia, and of acute injury to the eye tissue.

Such compositions are useful in treatment of pulmonary inflammation, such as that associated with viral infections and cystic fibrosis, and in bone resorption such as that associated with osteoporosis.

Such compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such compositions are useful in treatment of allergic rhinitis, respiratory distress syndrome, endotoxin shock syndrome and liver disease.

Such compositions are useful in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, bums, and trauma following surgical and dental procedures.

Such compositions are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such compositions are useful in treatment of angiogenesis-related disorders in a subject, for example to inhibit tumor angiogenesis. Such compositions are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemaginomas, angiofibroma of the nasopharynx and avascular necrosis of bone; and disorders of the female reproductive system such as endometriosis.

Such compositions are useful in prevention and treatment of benign and malignant tumors and neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such compositions can also be used to treat fibrosis that occurs with radiation therapy. Such compositions can be used to treat subjects having adenomatous polyps, including those with familial adenomatous polyposis (FAP). Additionally, such compositions can be used to prevent polyps from forming in patients at risk of FAP.

Such compositions inhibit prostanoid-induced smooth muscle contraction by inhibiting synthesis of contractile prostanoids and hence can be of use in treatment of dysmenorrhea, premature labor, asthma and eosinophil-related disorders. They also can be of use for decreasing bone loss particularly in postmenopausal women (i.e., treatment of osteoporosis), and for treatment of glaucoma.

Preferred uses for compositions of the invention are for treatment of rheumatoid arthritis and osteoarthritis, for pain management generally (particularly post-oral surgery pain, post-general surgery pain, post-orthopedic surgery pain, and acute flares of osteoarthritis), for treatment of Alzheimer's disease, and for colon cancer chemoprevention.

For treatment of rheumatoid arthritis or osteoarthritis, compositions of the invention can be used to provide a daily dosage of celecoxib of about 50 mg to about 1000 mg, preferably about 100 mg to about 600 mg, more preferably about 150 mg to about 500 mg, still more preferably about 175 mg to about 400 mg, for example about 200 mg. A daily dose of celecoxib of about 0.7 to about 13 mg/kg body weight, preferably about 1.3 to about 8 mg/kg body weight, more preferably about 2 to about 6.7 mg/kg body weight, and still more preferably about 2.3 to about 5.3 mg/kg body weight, for example about 2.7 mg/kg body weight, is generally appropriate when administered in a composition of the invention. The daily dose can be administered in one to about four doses per day, preferably one or two doses per day.

For treatment of Alzheimer's disease or cancer, compositions of the invention can be used to provide a daily dosage of celecoxib of about 50 mg to about 1000 mg, preferably about 100 mg to about 800 mg, more preferably about 150 mg to about 600 mg, and still more preferably about 175 mg to about 400 mg, for example about 400 mg. A daily dose of about 0.7 to about 13 mg/kg body weight, preferably about 1.3 to about 10.7 mg/kg body weight, more preferably about 2 to about 8 mg/kg body weight, and still more preferably about 2.3 to about 5.3 mg/kg body weight, for example about 5.3 mg/kg body weight, is generally appropriate when administered in a composition of the invention. The daily dose can be administered in one to about four doses per day, preferably one or two doses per day.

For pain management, compositions of the invention can be used to provide a daily dosage of celecoxib of about 50 mg to about 1000 mg, preferably about 100 mg to about 600 mg, more preferably about 150 mg to about 500 mg, and still more preferably about 175 mg to about 400 mg, for example about 200 mg. A daily dose of celecoxib of about 0.7 to about 13 mg/kg body weight, preferably about 1.3 to about 8 mg/kg body weight, more preferably about 2 to about 6.7 mg/kg body weight, and still more preferably about 2.3 to about 5.3 mg/kg body weight, for example about 2.7 mg/kg body weight, is generally appropriate when administered in a composition of the invention. The daily dose can be administered in one to about four doses per day. Administration at a rate of one 50 mg dose unit four times a day, one 100 mg dose unit or two 50 mg dose units twice a day or one 200 mg dose unit, two 100 mg dose units or four 50 mg dose units once a day is preferred.

For selective COX-2 inhibitory drugs other than celecoxib, appropriate doses can be selected by reference to the patent literature cited hereinabove.

In general, a celecoxib composition of the invention is preferably administered at a dose suitable to provide an average blood serum concentration of celecoxib of at least about 100 ng/ml in a subject over a period of about 24 hours after administration.

Contemplated compositions of the present invention provide a therapeutic effect as selective COX-2 inhibitory medications over an interval of about 12 to about 24 hours after oral administration. Preferred compositions provide such therapeutic effect over about 24 hours, enabling once-a-day oral administration.

Besides being useful for human treatment, compositions of the invention are useful for veterinary treatment of companion animals, exotic animals, farm animals, and the like, particularly mammals. More particularly, compositions of the invention are useful for treatment of COX-2 mediated disorders in horses, dogs and cats.

The present invention is further directed to a therapeutic method of treating a condition or disorder where treatment with a COX-2 inhibitory drug is indicated, the method comprising oral administration of a composition of the invention to a subject in need thereof. The dosage regimen to prevent, give relief from, or ameliorate the condition or disorder preferably corresponds to once-a-day or twice-a-day treatment, but can be modified in accordance with a variety of factors. These include the type, age, weight, sex, diet and medical condition of the subject and the nature and severity of the disorder. Thus, the dosage regimen actually employed can vary widely and can therefore deviate from the preferred dosage regimens set forth above.

Initial treatment can begin with a dose regimen as indicated above. Treatment is generally continued as necessary over a period of several weeks to several months or years until the condition or disorder has been controlled or eliminated. Subjects undergoing treatment with a composition of the invention can be routinely monitored by any of the methods well known in the art to determine effectiveness of therapy. Continuous analysis of data from such monitoring permits modification of the treatment regimen during therapy so that optimally effective doses are administered at any point in time, and so that the duration of treatment can be determined. In this way, the treatment regimen and dosing schedule can be rationally modified over the course of therapy so that the lowest amount of the composition exhibiting satisfactory effectiveness is administered, and so that administration is continued only for so long as is necessary to successfully treat the condition or disorder.

The present compositions can be used in combination therapies with opioids and other analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise use of a composition of the invention with one or more compounds selected from aceclofenac, acemetacin, e-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac (see The Merck Index, 12th Edition (1996), Therapeutic Category and Biological Activity Index, lists therein headed “Analgesic”, “Anti-inflammatory” and “Antipyretic”).

Particularly preferred combination therapies comprise use of a composition of the invention with an opioid compound, more particularly where the opioid compound is codeine, meperidine, morphine or a derivative thereof.

A celecoxib composition of the invention can also be administered in combination with a second selective COX-2 inhibitory drug, for example valdecoxib, rofecoxib, etc.

The compound to be administered in combination with celecoxib can be formulated separately from the celecoxib or co-formulated with the celecoxib in a composition of the invention. Where celecoxib is co-formulated with a second drug, for example an opioid drug, the second drug can be formulated in immediate-release, rapid-onset, sustained-release or dual-release form.

Compositions of the invention comprise a selective COX-2 inhibitory drug of low water solubility in association with one or more preferably non-toxic, pharmaceutically acceptable carriers, excipients and adjuvants (collectively referred to herein as “excipients”) suitable for oral administration. The excipients must be acceptable in the sense of being compatible with the other ingredients of the composition and must not be deleterious to the recipient. Compositions of the invention can be adapted for administration by any suitable oral route by selection of appropriate excipients and a dosage of the drug effective for the treatment intended. Accordingly, excipients employed can be solids, semi-solids and/or liquids. Compositions of the invention can be prepared by any well known technique of pharmacy that comprises admixing the components.

A celecoxib composition of the invention can be in the form of, for example, a tablet, a pill, a hard or soft capsule, a lozenge, a cachet, a dispensable powder, granules, a suspension, an elixir, a liquid, or any other form reasonably adapted for oral administration.

Compositions suitable for buccal or sublingual administration include, for example, lozenges comprising the selective COX-2 inhibitory drug in a flavored base, such as sucrose and acacia or tragacanth, and pastilles comprising the drug in an inert base such as gelatin and glycerin or sucrose and acacia.

Liquid dosage forms for oral administration include pharmaceutically acceptable suspensions, syrups, and elixirs containing inert diluents commonly used in the art, such as water. Such compositions may also comprise, for example, wetting agents, emulsifying and suspending agents, and sweetening, flavoring, and perfuming agents.

Solid unit dosage forms for oral administration contain the selective COX-2 inhibitory drug together with one or more excipients and are most conveniently formulated as tablets or capsules.

In general, such compositions are prepared by uniformly and intimately admixing the drug with a finely divided and/or liquid excipient carrier, and then, if necessary, encapsulating or shaping the product. For example, a tablet can be prepared by compressing or molding a powder or granules containing the drug together with one or more excipients. Compressed tablets can be prepared by compressing, in a suitable machine, a free-flowing composition, such as a powder or granules, comprising the drug optionally mixed with one or more binding agent(s), lubricant(s), inert diluent(s), wetting agent(s) and/or dispersing agent(s). Molded tablets can be made by molding, in a suitable machine, the powdered compound moistened with an inert liquid diluent.

Although a wide range of excipients can be used, a class of excipient common to all compositions of the present invention is that defined herein as a release-extending polymer, which can be a swellable or erodible polymer, or a polymer suitable for combining with a water-soluble polymer in a coating that becomes porous when placed in an aqueous medium. The sustained-release properties of compositions of the invention are in part or in whole attributable to the presence of such polymers as set out more fully hereinbelow.

Importantly, not all swellable or erodible polymers have release-extending properties. For example HPMCs of low viscosity (less than 100 cP) have now been found to be ineffective in slowing release of poorly water-soluble selective COX-2 inhibitory drugs. One of ordinary skill can readily determine if a swellable or erodible polymer is release-extending as defined herein, and thereby provides sustained-release characteristics to a formulation containing it, by standard dissolution tests known in the art. Non-limiting examples of standard dissolution tests can be found in the patents and publications listed below, each of which is individually incorporated herein by reference.

Above-cited U.S. Pat. No. 5,536,505.

Above-cited U.S. Pat. No. 5,523,095.

International Patent Publication No. WO 96/38174.

International Patent Publication No. WO 96/41617.

See also Lieberman et al., op. cit.

In a sustained-release composition of the invention, the drug is present as solid particles, herein termed “primary particles”, which are typically agglomerated, optionally with the aid of a binding agent, into larger aggregates or “secondary particles” such as granules or beads. When the term “particle size” is used herein, this term refers to the primary particles of celecoxib or other selective COX-2 inhibitory drug unless the context requires otherwise. Particle size is expressed herein as the percentage by weight of total particles that have a diameter smaller than a given reference diameter. For example, if a batch of a drug has a D₉₀ particle size of 60 μm, 90% of the particles in that batch have a diameter less than 60 μm. Although compositions of the invention are effective over a broad range of particle sizes, it has been discovered that reduction of particle size can improve bioavailability of a poorly water-soluble selective COX-2 inhibitory drug. Accordingly, the D₉₀ particle size of the drug is preferably less than about 200 μtm, more preferably less than about 100 μm, still more preferably less than about 75 μm, and still more preferably less than about 40 μm. For example, reducing the D₉₀ particle size of celecoxib from about 60 μm to about 30 μm can materially improve the bioavailability of the celecoxib in a composition of the invention.

Although solid unit dose compositions of the invention can be prepared, for example, by direct encapsulation or direct compression, they are preferably wet granulated prior to encapsulation or compression. Wet granulation, among other effects, densifies milled compositions resulting in improved flow properties, improved compression characteristics and easier metering or weight dispensing of the compositions for encapsulation or tableting. The secondary particle size arising from granulation (i.e., granule size) is not narrowly critical, it being important only that the average granule size is preferably such as to allow for convenient handling and processing and, for tablets, to permit formation of a directly compressible mixture that forms pharmaceutically acceptable tablets.

Desired tap and bulk densities of the granulation are normally about 0.3 g/ml to about 1.0 g/ml.

Tablets and capsules prepared according to the invention have desirable dissolution profiles in which drug release is slower at early time periods but continues longer than in the case of standard immediate-release compositions, as measured in standard dissolution tests. For example, the amount of the drug released from a composition of the invention 2 hours after commencement of such a test is significantly less than that released from a standard composition. Release of the drug from a composition of the invention continues for at least about 8 hours, in the case of preferred compositions at least about 18 hours, whereas release from a standard composition is typically complete within a significantly shorter time.

A composition having a dissolution profile in which substantially less than 50% of the drug contained therein is released in the first hour after placement in a dissolution medium is considered to be a sustained-release composition. Ideally, a sustained-release composition releases substantially less than about 50% of the drug one hour after placement in a dissolution medium and at least about 90% of the drug by 24 hours after placement in the dissolution medium. In contrast, immediate-release compositions typically release at least 50% of drug contained therein in the first hour after placement in a dissolution medium. Celecoxib tablets or capsules in accordance with one embodiment of the invention show about 5% to about 35% dissolution in 2 hours, about 10% to about 90% dissolution in 8 hours, and at least about 90% dissolution in 24 hours. Preferred celecoxib tablets and capsules of the invention show about 5% to about 25% dissolution in 2 hours, about 10% to about 80% dissolution in 8 hours, and at least about 90% dissolution in 24 hours. Most preferred celecoxib tablets of the present invention show about 5% to about 15% dissolution in 2 hours, about 20% to about 40% dissolution in 8 hours, and substantially complete dissolution in 24 hours.

To prepare tablets, a complete mixture in an amount sufficient to make a uniform batch of tablets is subjected to tableting in a conventional production scale tableting machine, for example a Carver press, at normal compression pressure (for example, about 1 kP to about 15 kP). Any tablet hardness convenient with respect to handling, manufacture, storage and ingestion may be employed. For 100 mg tablets, hardness is preferably at least about 4 kP, more preferably at least about 5 kP, and still more preferably at least about 6 kP. For 200 mg tablets, hardness is preferably at least about 7 kP, more preferably at least about 9 kP, and still more preferably at least about 11 kP. For 1000 mg tablets, hardness is preferably at least about 10 kP, more preferably at least about 12 kP, and still more preferably at least about 14 kP. The mixture, however, is not be compressed to such a degree that there is subsequent difficulty in achieving hydration when exposed to gastric fluid.

Tablet friability preferably is less than about 1.0%, more preferably less than 0.8%, and still more preferably less than about 0.5%, in a standard test.

As noted above, compositions of an embodiment of the invention comprise a selective COX-2 inhibitory drug such as celecoxib in a therapeutically or prophylactically effective amount, and a release-extending polymer. Preferred compositions further comprise one or more pharmaceutically acceptable excipients selected from the group consisting of diluents, disintegrants, binding agents, adhesives, wetting agents, lubricants, and anti-adherent agents. More preferably, such compositions are in the form of matrix compositions, particularly matrix tablets, or coated bead compositions, particularly coated bead capsules.

Through selection and combination of excipients, compositions can be provided exhibiting improved performance with respect to, among other properties, efficacy, bioavailability, clearance time, stability, compatibility of drug and excipients, safety, dissolution profile, disintegration profile and/or other pharmacokinetic, chemical and/or physical properties. Where the composition is formulated as a tablet, the combination of excipients selected provides tablets that can exhibit improvement, among other properties, in dissolution profile, hardness, crushing strength, and/or friability.

Compositions of the invention optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of α- and amorphous cellulose (e.g., Rexcel™) and powdered cellulose; calcium carbonate; glycine; bentonite; polyvinylpyrrolidone; and the like. Such diluents, if present, constitute in total about 5% to about 99%, preferably about 10% to about 85%, and more preferably about 20% to about 80%, of the total weight of the composition. The diluent or diluents selected preferably exhibit suitable flow properties and, where tablets are desired, compressibility.

Lactose and microcrystalline cellulose, either individually or in combination, are preferred diluents. Both diluents are chemically compatible with celecoxib. The use of extragranular microcrystalline cellulose (that is, microcrystalline cellulose added to a wet granulated composition after a drying step) can be used to improve hardness (for tablets) and/or disintegration time. Lactose, especially lactose monohydrate, is particularly preferred. Lactose typically provides compositions having suitable release rates of celecoxib, stability, pre-compression flowability, and/or drying properties at a relatively low diluent cost. It provides a high density substrate that aids densification during granulation (where wet granulation is employed) and therefore improves blend flow properties.

Compositions of the invention optionally comprise one or more pharmaceutically acceptable disintegrants as excipients, particularly for tablet formulations. Suitable disintegrants include, either individually or in combination, starches, including sodium starch glycolate (e.g., Explotab™ of PenWest) and pregelatinized corn starches (e.g., National™ 1551, National™ 1550, and Colorcon™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, locust bean, karaya, pectin and tragacanth gums.

Disintegrants may be added at any suitable step during the preparation of the composition, particularly prior to granulation or during a lubrication step prior to compression. Such disintegrants, if present, constitute in total about 0.2% to about 30%, preferably about 0.2% to about 10%, and more preferably about 0.2% to about 5%, of the total weight of the composition.

Croscarmellose sodium is a preferred disintegrant for tablet or capsule disintegration, and, if present, preferably constitutes about 0.2% to about 10%, more preferably about 0.2% to about 7%, and still more preferably about 0.2% to about 5%, of the total weight of the composition. Croscarmellose sodium confers superior intragranular disintegration capabilities to granulated compositions of the present invention.

Compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™). Such binding agents and/or adhesives, if present, constitute in total about 0.5% to about 25%, preferably about 0.75% to about 15%, and more preferably about 1% to about 10%, of the total weight of the composition.

Compositions of the invention optionally comprise one or more pharmaceutically acceptable wetting agents as excipients. Such wetting agents are preferably selected to maintain the selective COX-2 inhibitory drug in close association with water, a condition that is believed to improve bioavailability of the composition.

Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefosse), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the composition.

Wetting agents that are anionic surfactants are preferred. Sodium lauryl sulfate is a particularly preferred wetting agent. Sodium lauryl sulfate, if present, constitutes about 0.25% to about 7%, more preferably about 0.4% to about 4%, and still more preferably about 0.5% to about 2%, of the total weight of the composition.

Compositions of the invention optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. Such lubricants, if present, constitute in total about 0 1% to about 10%, preferably about 0.2% to about 8%, and more preferably about 0.25% to about 5%, of the total weight of the composition.

Magnesium stearate is a preferred lubricant used, for example, to reduce friction between the equipment and granulated mixture during compression of tablet formulations.

Suitable anti-adherents include talc, cornstarch, DL-leucine, sodium lauryl sulfate and metallic stearates. Talc is a preferred anti-adherent or glidant used, for example, to reduce formulation sticking to equipment surfaces and also to reduce static in the blend. Talc, if present, constitutes about 0.1% to about 10%, more preferably about 0.25% to about 5%, and still more preferably about 0.5% to about 2%, of the total weight of the composition.

Other excipients such as colorants, flavors and sweeteners are known in the pharmaceutical art and can be used in compositions of the present invention. Tablets can be coated, for example with an enteric coating, or uncoated. Compositions of the invention can further comprise, for example, buffering agents.

Sustained-release Matrix Tablets

An embodiment of the present invention is a composition comprising a therapeutically effective amount of a selective COX-2 inhibitory drug of low solubility, for example celecoxib, a substantial portion or all of which is distributed in a matrix comprising one or more pharmaceutically acceptable swellable or erodible polymers. In this embodiment the swellable polymers comprise HPMC having a viscosity, 2% in water, of about 100 to about 20,000 cP. Compositions of this embodiment of the invention are referred to for convenience herein as “matrix compositions”. When formulated as tablets, which are a preferred dosage form for this embodiment, such compositions are referred to herein as “matrix tablets”.

A matrix composition of the invention comprises HPMC in an amount sufficient to extend the release profile of the drug. Typically such an amount is about 0.1% to about 40%, preferably about 5% to about 30%, for example about 10%, of the composition by weight. Preferably the weight ratio of HPMC to the drug is about 1:1 to about 1:12, more preferably about 1:1 to about 1:6.

HPMCs vary in the chain length of their cellulosic backbone. This directly affects the viscosity of an aqueous dispersion of the HPMC. Viscosity is normally measured at a 2% by weight concentration of the HPMC in water. HPMCs having viscosity, 2% in water, of less than about 100 cP can be useful, for example as binding agents, but tend not to have useful release-extending properties for medicaments. Such HPMCs are said to have good binding properties and less desirable sustaining properties. The term “binding properties” herein refers to suitability as a binding agent for tablet production by wet granulation, wherein, for example, HPMC is dissolved in water for spraying on to dry powders to be granulated. The term “sustaining properties” herein refers to suitability as a release-extending matrix. HPMCs with good sustaining properties are typically too viscous for use as binding agents in wet granulation techniques. According to the present invention, the HPMC(s) used to form the matrix should have a viscosity, 2% in water, of about 100 to about 8,000 cP, preferably about 1000 to about 8,000 cP, for example about 4000 cP.

HPMCs also vary in the degree of substitution of available hydroxyl groups on the cellulosic backbone by methoxyl groups and by hydroxypropoxyl groups. With increasing hydroxypropoxyl substitution, the resulting HPMC becomes more hydrophilic in nature. It is preferred in matrix compositions of the invention to use HPMCs having about 15% to about 35%, more preferably about 19% to about 30%, and most preferably about 19% to about 24%, methoxyl substitution, and having about 3% to about 15%, more preferably about 4% to about 12%, and most preferably about 7% to about 12%, hydroxypropoxyl substitution.

HPMCs which are relatively hydrophilic in nature and are useful in compositions in the invention are illustratively available under the brand names Methocelm of Dow Chemical Co. and Metolose™ of Shin-Etsu Chemical Co. Examples of HPMCs of a low viscosity grade, generally unsuitable in compositions of the present invention except as binding agents, include Methocel™ E5, Methocel™ E15 LV, Methocel™ E50 LV, Methocel™ K100 LV and Methocel™ F50 LV, whose 2% by weight aqueous solutions have viscosities of 5 cP, 15 cP, 50 cP, 100 cP and 50 cP, respectively. Examples of HPMCs having medium viscosity include Methocel™ E4M and Methocel™ K4M, 2% by weight aqueous solutions of each of which have a viscosity of 4000 cP. Examples of HPMCs having high viscosity include Methocel™ E10M, Methocelm K15M and Methocel™ K100M, 2% by weight aqueous solutions of which have viscosities of 10,000 cP, 15,000 cP and 100,000 cP respectively. Various HPMC products are described in Anon. (1997) Formulating for Controlled Release with Methocel Premium Cellulose Ethers, Dow Chemical Co. The methoxyl and hydroxypropoxyl substitution type and content for selected HPMC products is provided in Table 1, below.

TABLE 1
Properties of selected HPMC products

| Methocel ™ E4MP | Nominal Viscosity, 2% in Water | 4,000 cP |
|---|---|---|
| (U.S. Pat. No. 2910) | Methoxyl, % | 28-30 |
| | Hydroxypropoxyl, % | 7-12 |
| Methocel ™ K4MP | Nominal Viscosity, 2% in Water | 4,000 cP |
| (U.S. Pat. No. 2208) | Methoxyl, % | 19-24 |
| | Hydroxypropoxyl, % | 7-12 |
| Methocel ™ E10MP | Nominal Viscosity, 2% in Water | 10,000 cP |
| (U.S. Pat. No. 2910) | Methoxyl, % | 28-30 |
| | Hydroxypropoxyl, % | 7-12 |
| Methocel ™ K15MP | Nominal Viscosity, 2% in Water | 15,000 cP |
| (U.S. Pat. No. 2208) | Methoxyl, % | 19-24 |
| | Hydroxypropoxyl, % | 7-12 |

An illustrative presently preferred HPMC with release-extending properties is one with substitution type 2208, denoting about 19% to about 24% methoxyl substitution and about 7% to about 12% hydroxypropoxyl substitution, and with a nominal viscosity, 2% in water, of about 4000 cP. A “controlled release” grade is especially preferred, having a particle size such that at least 90% passes through a 100-mesh screen. An example of a commercially-available HPMC meeting these specifications is Methocel™ K4M of Dow Chemical Co.

Without being bound by any particular hypothesis as to how the HPMC matrix according to the invention provides superior sustained-release characteristics, it is believed that upon oral ingestion and contact with gastrointestinal fluids, HPMC on or close to the tablet surface partially hydrates and thereby swells to form a gel layer having the active ingredient, e.g., celecoxib, distributed in a three-dimensional matrix therein. It is further believed that this outer three-dimensional gel matrix layer slows dissolution of the tablet. As the outer gel layer slowly dissolves, disperses or erodes, celecoxib is released from this layer into the gastrointestinal fluid where it is available for absorption. Meanwhile, hydration of the HPMC matrix gradually advances towards the center of the tablet, permitting further release of celecoxib over time by the same process hypothetically described above. Since the active ingredient is distributed throughout the tablet at a more or less uniform concentration throughout the HPMC matrix, a fairly constant amount of active ingredient can, according to the present non-limiting theory, be released per unit time in vivo by dissolution, dispersion or erosion of the outer portions of the tablet.

Overall release rate and consequently drug availability are dependent on the rate of diffusion of the drug through the outer gel layer and the rate of erosion of this layer of the tablet. Preferably T-90% (the time required for 90% drug release) in vivo is less than 24 hours, so that a clearance time exists whereby the tablet is suitable for once-a-day administration.

The process described below is an illustrative method to make celecoxib matrix tablets.

1. Dry Mixing: A mixer (e.g., a 60 liter Baker Perkins blender) is loaded with lactose, micronized celecoxib, microcrystalline cellulose (e.g., an Avicel™ product), HPMC (e.g., Methocel™ K4M), and a suitable binder (e.g., Pharmacoat™ 603), preferably in this order. These materials are mixed, for example for three minutes with a slow main blade setting and a slow chopper blade setting, to form a dry powder mixture.

2. Wet granulation: The dry powder mixture is wet granulated, conveniently in the same blender with the main blade and chopper blade on a fast speed setting. Water is added in an amount and at a rate appropriate to the amount of dry powder mixture, illustratively at about 1-1.5 kg/minute for about 3 minutes. The resulting wet granulated mixture is blended for an additional period of time to ensure uniform distribution of water in the granulation. The wet granulated mixture contains about 30% water by weight.

3. Drying: The wet granulated mixture is dried, for example in an Aeromatic fluid bed dryer with inlet air temperature set at about 60° C., to reduce the moisture content to about 1% to about 3% by weight. Moisture content of the granules can be monitored, for example using a Computrac Moisture Analyzer.

4. Dry screening: The resulting dry granules are milled and screened, for example by passing through a Fitzpatrick mill (D6A) with 20-mesh screen, knives forward and medium speed setting (1500-2500 rpm). The milled granules are collected, for example in a polyethylene bag.

5. Lubrication: The resulting screened granules are placed in a mixer, for example a Paterson-Kelley 2 cubic foot V-blender. Talc is added to the granules and the granules are blended for about 5 minutes. Magnesium stearate is then added to the granules and the granules are blended for about 3 minutes. The resulting lubricated granules are discharged from the blender, for example into a fiber drum lined with double polyethylene bags.

6. Compression: The lubricated granules are compressed, for example on a Korsch tablet press, to form tablets having a desired weight and hardness.

7. Preparation of coating suspension: Water is illustratively added to a stainless steel container and stirred by an electric mixer with a stainless steel impeller at slow speed to form a vortex. A suitable coating material, e.g., Opadry (white: YS-1-18027-A) in an amount of about 10% by weight, is slowly added to the vortex. The stirring speed is increased as necessary to disperse the Opadry in the water while avoiding formation of foam. Mixing continues for about 30 minutes or until all the coating material is dispersed and a homogeneous suspension is observed. The coating suspension is kept under constant slow stirring during the following coating step.

8. Coating: Any suitable coating equipment such as a Compulab Coater can be used to apply a desired amount of coating material, typically about 3% by weight, to the tablets. The coated tablets are discharged, for example into fiber drums lined with double polyethylene bags.

Sustained-release Coated Bead Capsules

Coated bead formulations of the present invention are preferably encapsulated, however, if desired, they can be tableted. It has been found that the demands of a sustained-released formulation are met surprisingly well by a preparation containing a large number of more or less discrete beads, pellets or granules (herein all encompassed by the term “beads”) comprising a selective COX-2 inhibitory drug of low water solubility, illustratively celecoxib, a substantial portion or all of which are coated with a barrier layer containing at least one polymer that is substantially insoluble in gastrointestinal fluid.

In one embodiment, the beads optionally contain pharmaceutically acceptable excipients such as lactose and microcrystalline cellulose and have a size of about 0.1 to about 1.0 mm, preferably about 0.18 to about 0.425 mm. The beads are prepared by conventional methods, for example comprising mixing and granulation of the drug with excipients, extrusion, spheronization, drying and sizing the particles to an acceptable size range.

In another embodiment, the beads have a core comprising a pharmaceutically acceptable excipient such as starch or sucrose, surrounded by one or more shells each comprising an inner drug-containing layer and an outer polymer barrier layer. Beads according to this embodiment are preferably about 0.5 mm to about 2 mm, more preferably about 0.5 mm to about 1 mm, in diameter.

In a barrier layer preferred according to the present invention, the beads containing the drug and excipients are coated with one or more polymers selected from HPMC, hydroxypropylcellulose, hydroxyethylcellulose, methylhydroxyethylcellulose, methylcellulose, ethylcellulose (e.g., Surelease™ of Colorcon), cellulose acetate, sodium carboxymethylcellulose, polymers and copolymers of acrylic acid and methacrylic acid and esters thereof (e.g., Eudragit™ RL, Eudragit™ RS, Eudragit™ L100, Eudragit™ S100, Eudragit™ NE), polyvinylpyrrolidone and polyethylene glycols. The polymers can be combined with water-soluble substances such as sugar, lactose and salts to form a coating providing a pH-independent or pH-dependent release rate.

Eudragit™ of Rohm Pharma is a trade name applied to a range of products useful for film coating of sustained-release particles. These products are of varying solubility in gastrointestinal fluids. Eudragit™ RL and Eudragit™ RS are copolymers synthesized from acrylic and methacrylic esters with a low content of quaternary ammonium groups. Eudragit™ RL and Eudragit™ RS differ in the mole ratios of such ammonium groups to the remaining neutral (meth)acrylic acid esters (1:20 and 1:40 respectively). Eudragit™ NE is an aqueous dispersion of a neutral copolymer based on ethyl acrylate and methyl methacrylate. Characteristics of Eudragit™ polymers are described in Eudragit: Sustained-release Formulations for Oral Dosage Forms, Rohm Basic Info 2.

Ethylcellulose, available as an aqueous dispersion, for example under the trade name Surelease™, is another suitable material which is available in different grades and in special qualities for preparing barrier coatings. According to the invention it is preferred to use ethylcellulose having a viscosity of about 5 cP to about 15 cP, but other types of cellulose-based polymers can be used. It is especially preferred to use ethylcellulose in combination with HPMC.

The coating procedure can be performed by conventional means employing, for example, spraying equipment, a fluidized bed and equipment for drying and size fractionating. The liquid used in the coating procedure contains one or more barrier layer forming components and one or more solvents, such as ethanol, acetone, methyl isobutyl ketone (MIBK), water and others well known in this technical field. The coating liquid can be in the form of a solution, a dispersion, an emulsion or a melt, depending on the specific nature of the coating constituents.

Plasticizers and pigments can optionally be used to modify the technical properties or change the permeability of the coating. The coating preferably has virtually pH independent permeability properties throughout a pH range of 1.0 to 7.0. At higher pH a reduction in the release rate of certain drugs such as celecoxib may be observed but this is not due to the properties of the polymeric layer but to reduced solubility of the drug at high pH values.

An illustrative suitable coating composition according to the invention comprises ethylcellulose and HPMC together with a plasticizer such as triethyl citrate or coconut oil. A specific example of such a coating composition contains 90% polymer consisting of ethylcellulose and HPMC in a weight ratio of 55:35 to 80:10, with 10% triethyl citrate.

Each coated bead containing a selective COX-2 inhibitory drug represents an individual controlled release unit, releasing the drug at a predetermined rate, preferably independent of its position in the gastrointestinal tract. Coated beads according to the invention can be used in different types of dosage forms such as gelatin capsules, compressed tablets or sachets.

The drug, illustratively celecoxib, can be formulated in a sustained-release coated bead preparation according to the present invention by the following procedures. Overall dissolution rate and drug availability are dependent on the rate of drug diffusion through the coating and/or the rate of erosion of the coating.

The process described below is an illustrative method to make celecoxib coated beads.

1. Mixing and granulating: Celecoxib and diluents, preferably lactose and/or microcrystalline cellulose, are mixed and granulated by the following illustrative process. Celecoxib is added to a mixture of lactose and microcrystalline cellulose (e.g., Avicel™ PH-101 and/or Avicel™ RC-581 or Avicel™ RC-591) in a total amount of 1000-4000 g and are dry-mixed in a high shear mixer (e.g., Niro-Fielder mixer) at a high mixing speed for 2-5 minutes. Water (300-700 g) is added and the mass is granulated for 2-5 minutes at high speed.

2. Extrusion: Extrusion of the resulting material can be performed for example in a NICA E-140 extruder (Lejus Medical AB, Sweden) through a perforated screen with drilled orifices of 0.25-1.0 mm diameter. The speed of the agitator and the feeder are preferably set on the lowest values.

3. Spheronization: Spheronization of the resulting extrudate can be conducted in a NICA marumerizer (Ferro Mecano AB, Sweden). The speed of the marumerizer plate is preferably adjusted to 500-10,000 rpm. The spheronization continues for 2-10 minutes, with about 1000 g wet extrudate on the plate at each run.

4. Drying: Drying of the resulting spheronized beads can be performed in a fluidized bed dryer (e.g., Aeromatic AG, West Germany) at an inlet temperature of 50-90° C. A net device can be placed in the top of the fluidized bed to avoid loss of beads to the cyclone output. The batch is preferably divided into sub-batches of 200-800 g. Each sub-batch is dried for 10-60 minutes at an air volume of 100-400 m³/h in order to obtain individual beads rather than aggregates. If necessary, the sub-batches are then mixed and the whole batch dried for 5-30 minutes to an end product temperature of 40-60° C. A yield of dry beads of 1600-2000 g can be expected.

5. Sizing: Sizing of the resulting dry beads can be performed using analytical sieves. Two sieves are selected from a set of sieve sizes, for example of 850 μm, 600 μm, 425 μm, 300 μm, 250 lm and 180 μm. A preferred pair of sieves for sizing beads of the present invention is 425 μm and 180 μm.

6. Coating: Celecoxib beads manufactured as above can be coated with swellable or erodible polymers to prepare sustained-release formulations of the present invention. For example, Surelease™ or Eudragit™ RS can be applied as a 10-20% by weight solids dispersion, using spray coating equipment (e.g., Wurster). The spray gun is mounted at a height of 0.25 cm to 5 cm over the bottom of the bed. Celecoxib beads prepared as above are loaded and preferably pre-heated. The coating is applied using the following process parameters: atomizing pressure 1.0-3.0 bar, air temperature 50-80° C., air velocity 100-400 m³/h and solution flow about 10-80 ml/minute.

7. Encapsulating. The coated beads manufactured as above, optionally together with uncoated beads, are encapsulated by a conventional encapsulation process.

### EXAMPLES

Dissolution Assay

Drug release profiles of tablets and coated beads were evaluated in a standard in vitro USP dissolution assay under the following conditions. USP apparatus II paddles were used to stir a dissolution medium (1 liter water containing 1% sodium dodecyl sulfate) at a speed of 50 rpm and a temperature of 37° C. The medium was then filtered through 10 mm Van-Kel filters. Samples were analyzed via UV detection.

Examples of Celecoxib Matrix Tablets

Matrix tablets of celecoxib, Examples M4 to M21, were prepared having components as shown in Table 2 below. Compositions of the tablets are shown in Table 3 (M4 to M11) and Table 4 (M12 to M21) below.

The tablets were prepared by the following procedure. Lactose, micronized celecoxib, Avicel™, Methocel™ K4M and Pharmacoat™ 603 were added in this order to a 60 L Baker Perkins blender, and mixed for 3 minutes with the main blade on the slow main blade setting and the chopper blade on the slow chopper blade setting. About 3.1 kg of USP water was added over a period of about 3 minutes using an Aeromatic water pump, with the main blade and chopper blade of the blender on the fast speed setting. The resulting wet granulated mixture, about 31% by weight water, was blended for an additional minute to ensure uniform distribution of the water in the granulation, and was then placed in an Aeromatic fluid bed dryer with inlet air temperature set at about 60° C. Drying in the fluid bed dryer continued until moisture content of the granules was reduced to 1-3% by weight, as monitored using a Computrac Moisture Analyzer. The dried granules were screened by passing through a Fitzpatrick mill (D6A) with 20-mesh screen, knives forward and medium speed setting (1500-2500 rpm), and were then collected in a polyethylene bag. The resulting milled and screened granules were placed in a Paterson-Kelley 50 liter V-blender. Talc was placed on top of the granules and the granules were blended for 5 minutes. Magnesium stearate was then placed on top of the granules and the granules were blended for a further 3 minutes before being discharged into a fiber drum lined with double polyethylene bags. The resulting lubricated granules were compressed on a Korsch tablet press to form tablets of desired weight (333.3 mg) and hardness (11-13 kP), using 9 mm round standard concave tooling. A 10% Opadry (white: YS-1-18027-A) coating suspension was prepared and applied using a Compulab Coater with 36-inch coating pan and one spray gun. The atomization air pressure was set at 310 kPa. The tablets were weighed and the amount of coating suspension required to be sprayed in order to give 3% tablet weight gain was determined. The tablets were loaded into the pan and the air flow set to 19 m³/minute. The tablets were allowed to warm for approximately 10 minutes by jogging the pan every two minutes. The inlet air temperature was set at 65° C. The exhaust temperature obtained was about 45° C. The spray rate was set at 50 g/min with the pan rotating at 10 rpm. Pan rotation continued for an additional two to five minutes after the full amount of coating suspension had been sprayed. The tablets were allowed to cool for 10 minutes and the pan was jogged every two minutes during cooling. The resulting coated tablets were discharged from the coating pan into fiber drums lined with double polyethylene bags.

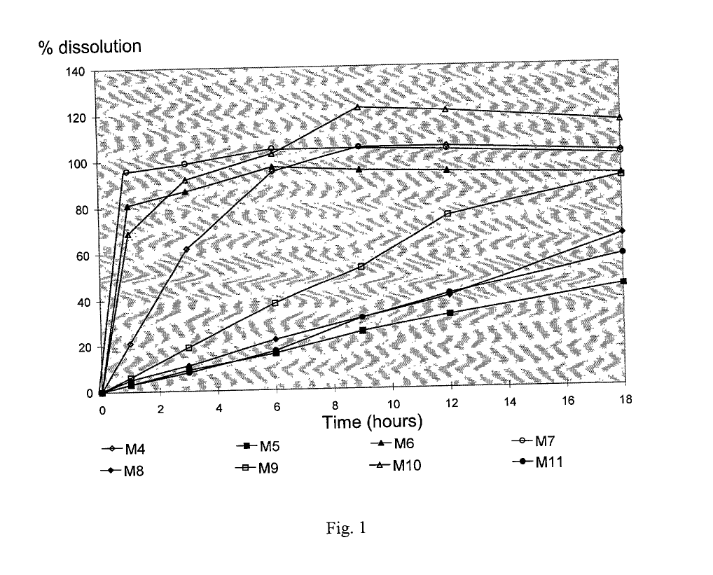

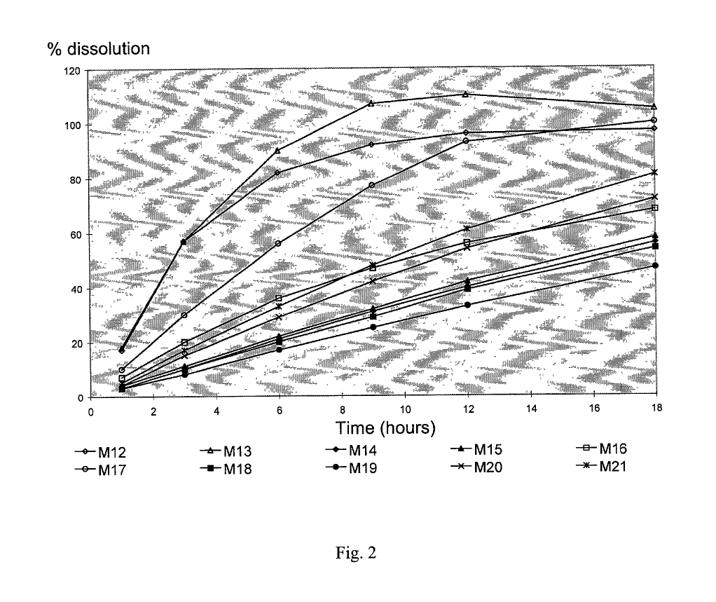

Celecoxib release profiles of these tablets were evaluated in the standard in vitro USP dissolution assay described above. Dissolution data from these studies are shown graphically in FIGS. 1 and 2.

TABLE 2
Celecoxib sustained-release matrix tablets of Examples M4 to M11:
components and composition

| Function | Component | Composition (%) |
|---|---|---|
| Drug | celecoxib | 20-50 |
| Diluent | Avicel ™ or lactose | q.s. |
| Swellable polymer | Methocel ™ E4M | 10-40 |
| | Methocel ™ E10M | |
| | Methocel ™ K4M | |
| | Methocel ™ K15M | |
| Binder | Pharmacoat ™ 603 | 3.0 |
| Glidant | talc | 1.0 |
| Lubricant | magnesium stearate | 0.5 |

TABLE 3
Composition (%) of Tablets of Examples M4 to M11

| Example | M4 | M5 | M6 | M7 | M8 | M9 | M10 | M11 |
|---|---|---|---|---|---|---|---|---|
| celecoxib | 20.0 | 20.0 | 20.0 | 20.0 | 50.0 | 50.0 | 50.0 | 50.0 |
| lactose hydrous | 65.5 | — | — | 65.5 | 5.5 | — | — | 5.5 |
| Avicel ™ | — | 35.5 | 35.5 | — | — | 35.5 | 35.5 | — |
| PH 101 | | | | | | | | |
| Methocel ™ | 10.0 | | | | 40.0 | | | |
| E4M | | | | | | | | |
| Methocel ™ | | 40.0 | | | | 10.0 | | |
| K4M | | | | | | | | |
| Methocel ™ | | | 40.0 | | | | 10.0 | |
| E10M | | | | | | | | |
| Methocel ™ | | | | 10.0 | | | | 40.0 |
| K15M | | | | | | | | |
| Pharmacoat ™ | 3.0 | 3.0 | 3.0 | 3.0 | 3.0 | 3.0 | 3.0 | 3.0 |
| 603 | | | | | | | | |
| talc | 1.0 | 1.0 | 1.0 | 1.0 | 1.0 | 1.0 | 1.0 | 1.0 |
| magnesium | 0.5 | 0.5 | 0.5 | 0.5 | 0.5 | 0.5 | 0.5 | 0.5 |
| stearate | | | | | | | | |

TABLE 4
Composition (%) of tablets of Examples M12 to M21

| Example | M12 | M13 | M14 | M15 | M16 | M17 | M18 | M19 | M20 | M21 |
|---|---|---|---|---|---|---|---|---|---|---|
| celecoxib | 40.0 | 40.0 | 40.0 | 40.0 | 60.0 | 60.0 | 60.0 | 60.0 | 50.0 | 50.0 |
| lactose hydrous | — | 50.5 | — | 20.5 | — | 30.5 | — | 0.5 | 12.75 | 12.75 |
| Avicel ™ PH 101 | 50.5 | — | 20.5 | — | 30.5 | — | 0.5 | — | 12.75 | 12.75 |
| Methocel ™ K4M | 5.0 | 5.0 | 35.0 | 35.0 | 5.0 | 5.0 | 35.0 | 35.0 | 20.0 | 20.0 |
| Pharmacoat ™ 603 | 3.0 | 3.0 | 3.0 | 3.0 | 3.0 | 3.0 | 3.0 | 3.0 | 3.0 | 3.0 |
| talc | 1.0 | 1.0 | 1.0 | 1.0 | 1.0 | 1.0 | 1.0 | 1.0 | 1.0 | 1.0 |
| magnesium stearate | 0.5 | 0.5 | 0.5 | 0.5 | 0.5 | 0.5 | 0.5 | 0.5 | 0.5 | 0.5 |

In general, compositions prepared using HPMC having a viscosity, 2% in water, of 4000 cP exhibited superior sustained-release dissolution profiles to those prepared using higher viscosity HPMC (10,000 or 15,000 cP). In general, compositions containing 10% HPMC exhibited superior sustained-release dissolution profiles to those containing 40% HPMC. See FIG. 1, wherein the most desirable dissolution profiles are exhibited by the composition of Example M9, which contains 10% Methocel™ K4M and the composition of Example M4, which contains 10% Methocel™ E4M. Example M9 exhibits slower release than Example M4.

FIG. 2 indicates that when the selected HPMC is Methocel™ K4M, release rate is inversely related to HPMC content. Compare, for example, compositions having 5% HPMC (Examples M12, M13, M16 and M17) with those having 20% HPMC (Examples M20 and M21) or 35% HPMC (Examples M14, M15, M18 and M19).

Table 5 presents calculated values of T-75% and T-90% (time in hours to reach 75% and 90% dissolution respectively) for the compositions of Examples M4 to M21.

TABLE 5
T-75% and T-90% for celecoxib matrix tablets

| Example | T-75% (h) | T-90% (h) |
|---|---|---|
| M4 | 4.2 | 5.5 |
| M5 | 30.9 | 37.1 |
| M6 | 0.9 | 3.9 |
| M7 | 0.8 | 0.9 |
| M8 | 20.5 | 24.7 |
| M9 | 12 | 17.6 |
| M10 | 1.1 | 2.8 |
| M11 | 23.7 | 28.4 |
| M12 | 5.2 | 7.0 |
| M13 | 4.6 | 6.0 |
| M14 | 24.1 | 28.9 |
| M15 | 23.4 | 28.1 |
| M16 | 20.0 | 24.1 |
| M17 | 8.7 | 11.4 |
| M18 | 25.0 | 30.0 |
| M19 | 28.8 | 34.6 |
| M20 | 18.8 | 22.5 |
| M21 | 16.2 | 20.0 |

Examples of Celecoxib Coated Bead Capsules

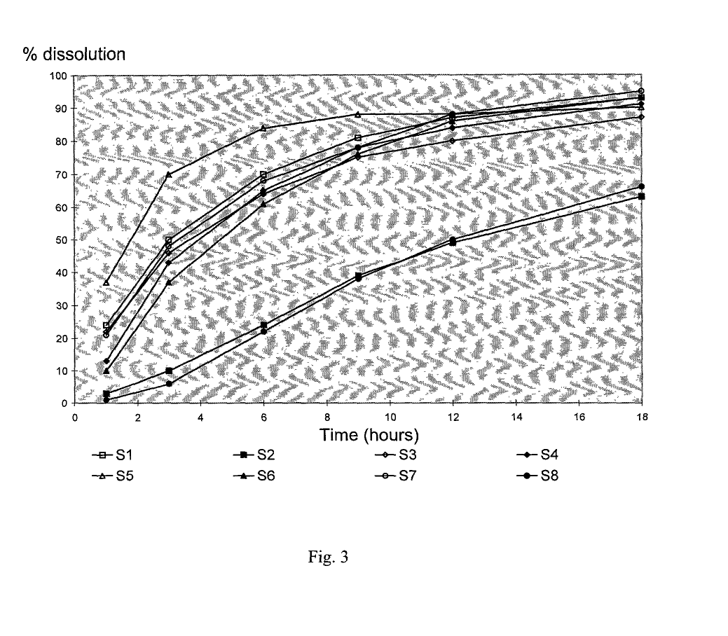

Coated bead capsules of celecoxib, Examples S1 to S8 having components as shown in Table 6 below and compositions as shown in Table 7 below, were prepared by the method described above. Celecoxib release profiles of these coated beads were evaluated in the standard in vitro USP dissolution assay described above. Dissolution data from these studies are shown graphically in FIG. 3.

TABLE 6
Celecoxib sustained-release coated bead capsules of Examples
S1 to S8: components and composition

| | | Composition |
|---|---|---|
| Function | Component | (% excluding coating) |
| Active | celecoxib | 50 |
| Diluent | Avicel ™ PH 101 | 50 |
| | Avicel ™ RC 581 | (total) |
| | lactose | |
| Coating | Surelease ™ | 3-15 |
| | Eudragit ™ RS | |

TABLE 7
Composition (%) of capsules of Examples S1 to S8

| Example | S1 | S2 | S3 | S4 | S5 | S6 | S7 | S8 |
|---|---|---|---|---|---|---|---|---|
| celecoxib | 50.0 | 50.0 | 50.0 | 50.0 | 50.0 | 50.0 | 50.0 | 50.0 |
| Avicel ™ | 50.0 | 50.0 | — | — | 25.0 | 25.0 | — | — |
| PH 101 | | | | | | | | |
| Avicel ™ | — | — | 50.0 | 50.0 | — | — | 25.0 | 25.0 |
| RC 581 | | | | | | | | |
| lactose | — | — | — | — | 25.0 | 25.0 | 25.0 | 25.0 |
| Surelease ™ | 3.0 | — | 15.0 | — | 15.0 | — | 3.0 | — |
| Eudragit ™ | — | 15.0 | — | 3.0 | — | 3.0 | | 15.0 |
| RS | | | | | | | | |

Table 8 presents calculated values of T-75% and T-90% (time in hours to reach 75% and 90% dissolution respectively) for the compositions of Examples SI to S8.

TABLE 8
T-75% and T-90% for celecoxib coated bead capsules

| Example | T-75% (h) | T-90% (h) |
|---|---|---|
| S1 | 7.4 | 15.0 |
| S2 | 21.4 | 25.7 |
| S3 | 9.0 | 18.8 |
| S4 | 8.3 | 17.1 |
| S5 | 4.1 | 18.0 |
| S6 | 8.8 | 15.4 |
| S7 | 8.1 | 13.7 |
| 58 | 20.4 | 24.3 |

Examples of Valdecoxib Matrix Tablets

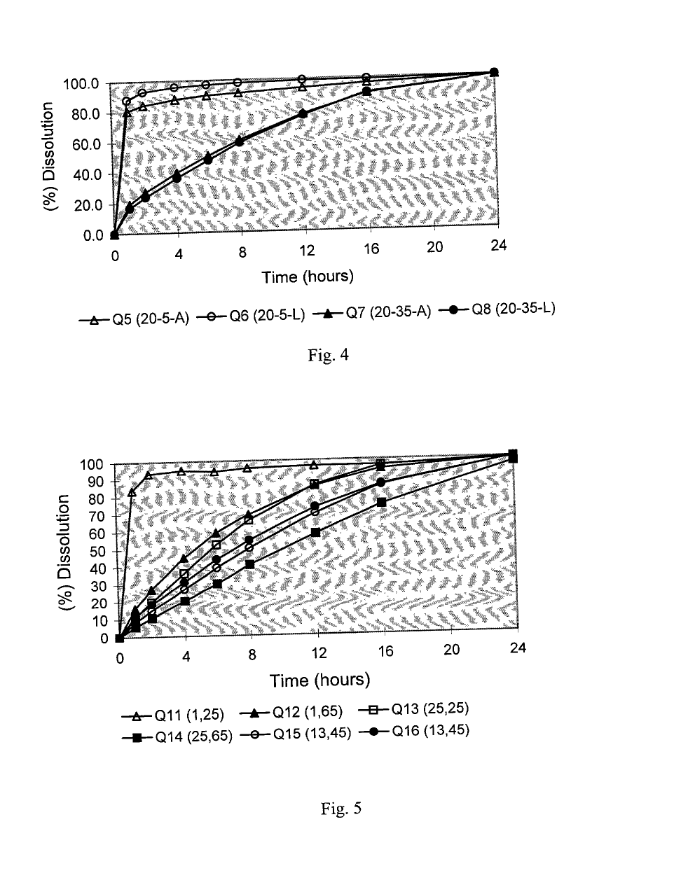

Matrix tablets of valdecoxib were first prepared by direct compression and displayed poor flowability and compression characteristics. The wet granulation method described above for celecoxib was subsequently used to produce additional valdecoxib tablets, Examples Q5 to Q8 and Q11 to Q29. Compositions of these tablets are shown in Table 9 (Q5 to Q8), Table 10 (Q11 to Q16), Table 11 (Q17 to Q20), and Table 12 (Q21 to Q29), below. Physical characteristics of these tablets are shown in Table 13, below. Valdecoxib release profiles of tablets Q5 to Q8 and Q11 to Q20 were evaluated in the standard in vitro USP dissolution assay described above. Dissolution data from these studies are shown graphically in FIGS. 4 and 5.

TABLE 9
Composition (%) of valdecoxib matrix tablets

| Formulation No. | Q5 | Q6 | Q7 | Q8 |
|---|---|---|---|---|
| valdecoxib | 20.0 | 20.0 | 20.0 | 20.0 |
| Avicel ™ PH 302 | 51.7 | 10.0 | 21.7 | 10.0 |
| lactose | 20.0 | 61.7 | 20.0 | 31.7 |
| Methocel ™ K4M | 5.0 | 5.0 | 35.0 | 35.0 |
| Aerosil ™ 200 | 0.5 | 0.5 | 0.5 | 0.5 |
| talc | 2.5 | 2.5 | 2.5 | 2.5 |
| magnesium stearate | 0.3 | 0.3 | 0.3 | 0.3 |

TABLE 10
Composition (%) of valdecoxib matrix tablets

| Formulation No. | Q11 | Q12 | Q13 | Q14 | Q15 | Q16 |
|---|---|---|---|---|---|---|
| celecoxib | 1.0 | 1.0 | 25.0 | 25.0 | 13.0 | 13.0 |
| lactose | 60.5 | 20.5 | 36.5 | 0.0 | 28.5 | 28.5 |
| Avicel ™ PH 302 | 10.0 | 10.0 | 10.0 | 10.0 | 10.0 | 10.0 |
| Methocel ™ K4M | 25.0 | 65.0 | 25.0 | 65.0 | 45.0 | 45.0 |
| Pharmacoat ™ 603 | 3.0 | 3.0 | 3.0 | 3.0 | 3.0 | 3.0 |
| magnesium stearate | 0.5 | 0.5 | 0.5 | 0.5 | 0.5 | 0.5 |

TABLE 11
Composition (%) of valdecoxib matrix tablets

| Formulation No. | Q17 | Q18 | Q19 | Q20 |
|---|---|---|---|---|
| valdecoxib | 5.0 | 5.0 | 5.0 | 5.0 |
| lactose | 45.5 | 49.5 | 33.5 | 17.5 |
| Avicel ™ PH 302 | 10.0 | 10.0 | 10.0 | 10.0 |
| Methocel ™ K100LV | 35.0 | — | — | — |
| Methocel ™ K4M Premium | — | 31.0 | 47.0 | 63.0 |
| Pharmacoat ™ 603 | 4.0 | 4.0 | 4.0 | 4.0 |
| magnesium stearate | 0.5 | 0.5 | 0.5 | 0.5 |

TABLE 12
Composition (%) of valdecoxib matrix tablets

| Formulation No. | Q21 | Q22 | Q23 | Q24 | Q25 | Q26 | Q27 | Q28 | Q29 |
|---|---|---|---|---|---|---|---|---|---|
| valdecoxib | 5.0 | 5.0 | 5.0 | 5.0 | 2.5 | 1.25 | 2.5 | 1.25 | 1.25 |
| lactose | 46.3 | 46.3 | 18.3 | 46.3 | 48.8 | 50.05 | 48.8 | 50.05 | 48.05 |
| Methocel ™ 100LV | 33.2 | 7.0 | — | 7.0 | 7.0 | 7.0 | — | — | — |
| Methocel ™ K4M | 1.8 | 28.0 | 63.0 | 28.0 | 28.0 | 28.0 | 35.0 | 35.0 | 37.0 |
| Pharmacoat ™ 603 | 3.5 | 3.5 | 3.5 | 3.5 | 3.5 | 3.5 | 3.5 | 3.5 | 3.5 |
| Avicel ™ PH 302 | 10.0 | 10.0 | 10.0 | 10.0 | 10.0 | 10.0 | 10.0 | 10.0 | 10.0 |
| magnesium stearate | 0.2 | 0.2 | 0.2 | 0.2 | 0.2 | 0.2 | 0.2 | 0.2 | 0.2 |

TABLE 13
Physical characteristics of valdecoxib matrix tablets

| | Average | Average | Average | | Bulk |
|---|---|---|---|---|---|
| Formulation | Weight | Thickness | Hardness | Friability | Density |
| No. | (mg) | (mm) | (kP) | (%) | (g/ml) |
| Q5 | 245.1 | 4.205 | 8.28 | 0.213 | 0.420 |
| Q6 | 244.8 | 4.107 | 8.55 | 0.317 | 0.430 |
| Q7 | 256.6 | 4.409 | 10.39 | 0.214 | 0.384 |
| Q8 | 256.8 | 4.300 | 13.32 | 0.164 | 0.385 |
| Q11 | 201.0 | 3.414 | 9.07 | 0.260 | 0.420 |
| Q12 | 201.7 | 3.754 | 8.14 | 0.238 | 0.313 |
| Q13 | 200.0 | 3.457 | 11.56 | 0.161 | 0.448 |
| Q14 | 203.0 | 3.776 | 10.83 | 0.264 | 0.345 |
| Q15 | 198.1 | 3.508 | 11.67 | 0.274 | 0.367 |
| Q16 | 200.8 | 3.695 | 9.25 | 0.361 | 0.349 |
| Q17 | 197.9 | 3.349 | 9.00 | 0.30 | 0.442 |
| Q18 | 203.4 | 3.476 | 9.76 | 0.28 | 0.426 |
| Q19 | 202.6 | 3.597 | 9.29 | 0.42 | 0.345 |
| Q20 | 199.6 | 3.698 | 7.65 | 0.40 | 0.342 |

Pharmacokinetic Properties

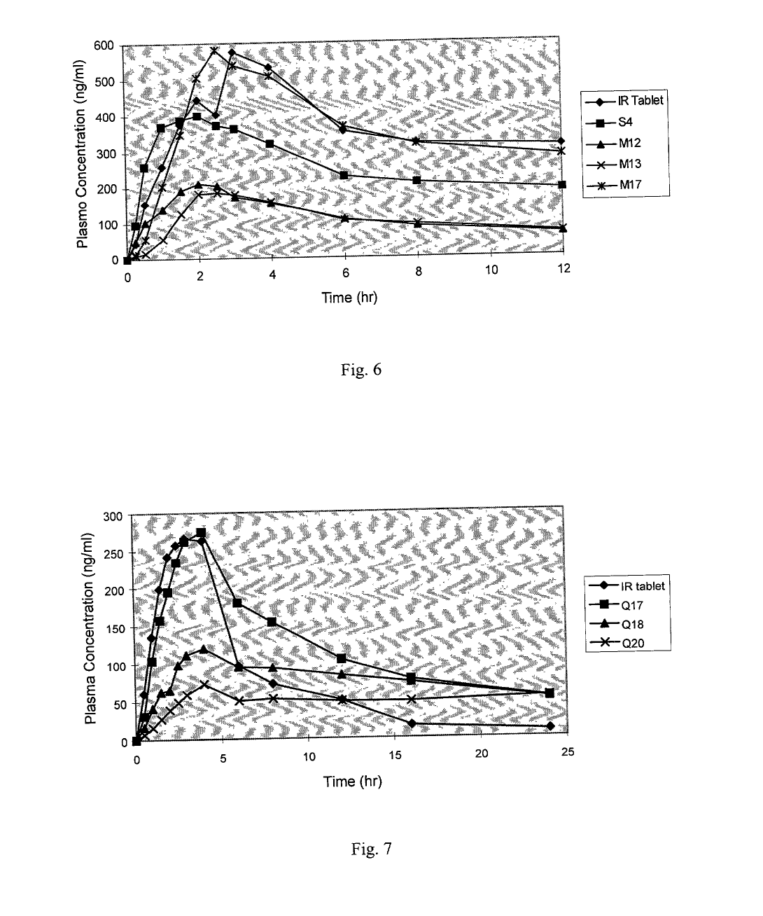

A study was performed to determine pharmacokinetic properties of the celecoxib formulations of Examples S4, M12, M13 and M17 in comparison to an immediate-release celecoxib tablet formulation, in 4 male and 4 female beagle dogs in a nonrandomized crossover design. Celecoxib was administered at a dose of 5 mg/kg. Venous blood was collected pre-dose, and at 1, 1.5, 2, 2.5, 3, 4, 6, 8, 12 and 24 hours after oral dose administration. Plasma was separated from blood by centrifugation at 3000 G and samples were stored at −20° C. until analysis. Concentrations of celecoxib in plasma were determined using an HPLC assay. Results are shown in FIG. 6.

Additionally, a study was performed in order to determine pharmacokinetic properties of the valdecoxib formulations of Examples Q17, Q18, Q 19 and Q20 in comparison to an immediate-release valdecoxib tablet formulation, in 23 beagle dogs. Valdecoxib was administered at a dose of 20 mg per day. Venous blood was collected pre-dose, and at 0.5, 1, 1.5, 2, 2.5, 3, 4, 6, 8, 12 and 24 hours after oral dose administration. Plasma was separated from blood by centrifugation at 3000 G and samples were stored at −20° C. until analysis. Concentrations of valdecoxib in plasma were determined using an HPLC assay. Results are shown in FIG. 7.

What is claimed is:

1. An orally deliverable pharmaceutical composition comprising a therapeutically effective amount of a selective cyclooxygenase-2 inhibitory drug of low water solubility and one or more pharmaceutically acceptable polymers, wherein the composition provides an in vitro sustained-release dissolution profile following placement in a standard dissolution medium exhibiting
(a) release of about 5% to about 35% of the drug 2 hours after said placement;
(b) release of about 10% to about 85% of the drug 8 hours after said placement; and
(c) release of about 30% to about 90% of the drug 18 hours after said placement.

2. The composition of claim 1 wherein said polymers are swellable or erodible polymers.

3. The composition of claim 1 wherein said polymers are release-extending polymers.

4. The composition of claim 1 exhibiting a time to reach 75% release of the drug of about 4 to about 18 hours after said placement.

5. The composition of claim 1 exhibiting a time to reach 90% release of the drug of about 5 to about 20 hours after said placement.

6. The composition of claim 1 exhibiting at least one of
(a) release of about 5% to about 25% of the drug 2 hours after said placement;
(b) release of about 10% to about 80% of the drug 8 hours after said placement; or
(c) release of about 75% to about 90% of the drug 18 hours after said placement.

7. The composition of claim 1 exhibiting
(a) release of about 5% to about 25% of the drug 2 hours after said placement;
(b) release of about 10% to about 80% of the drug 8 hours after said placement; and
(c) release of about 75% to about 90% of the drug 18 hours after said placement.

8. The composition of claim 1 wherein the selective cycloxygenase-2 inhibitory drug has the formula 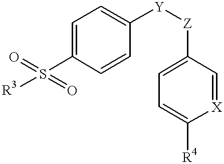
where R³ is a methyl or amino group, R⁴ is hydrogen or a C₁₋₄ alkyl or alkoxy group, X is N or CR⁵ where R⁵ is hydrogen or halogen, and Y and Z are independently carbon or nitrogen atoms defining adjacent atoms of a five- to six-membered ring that is unsubstituted or substituted at one or more positions with oxo, halo, methyl or halomethyl groups.

9. The composition of claim 8 wherein the five- to six-membered ring is selected from cyclopentenone, furanone, methylpyrazole, isoxazole and pyridine rings substituted at no more than one position.

10. The composition of claim 1 wherein the selective cyclooxygenase-2 inhibitory drug is selected from celecoxib, deracoxib, valdecoxib, rofecoxib, 5-chloro-3-(4-methylsulfonyl)phenyl-2-(2-methyl-5-pyridinyl)pyridine, 2-(3,5-difluorophenyl)-3-[4-(methylsulfonyl)phenyl]-2-cyclopenten-1-one and (S)-6,8-dichloro-2-(trifluoromethyl)-2H-1-benzopyran-3-carboxylic acid.

11. The composition of claim 1 wherein the selective cyclooxygenase-2 inhibitory drug is selected from celecoxib and valdecoxib.

12. The composition of claim 1 wherein the selective cyclooxygenase-2 inhibitory drug is celecoxib.

13. The composition of claim 12 that comprises one or more dose units each having about 10 mg to about 1000 mg of celecoxib.

14. The composition of claim 17 wherein the amount of celecoxib in each dose unit is about 100 mg to about 200 mg.

15. A composition of claim 1 that is suitable for providing therapeutically or prophylactically effective inhibition of cyclooxygenase-2 when orally administered to a subject once a day.

16. A composition of claim 1 comprising one or more dose units in a form of discrete solid articles.

17. The composition of claim 26 wherein said articles are tablets or capsules.

18. The composition of claim 1 further comprising one or more additional pharmaceutically acceptable excipients selected from lubricants, binding agents, glidants, dyes, fillers and extenders.

19. An orally deliverable pharmaceutical composition comprising a therapeutically effective amount of a selective cyclooxygenase-2 inhibitory drug of low water solubility, a substantial portion or all of said compound being distributed in a matrix comprising hydroxypropylmethylcellulose having a nominal viscosity, 2% in water, of about 100 to about 8,000 cP.

20. The composition of claim 19 wherein the hydroxypropylmethylcellulose is present in an amount of about 0.1% to about 40% by weight.

21. The composition of claim 19 wherein the hydroxypropylmethylcellulose is present in an amount of about 5% to about 30% by weight.

22. The composition of claim 19 wherein the hydroxypropylmethylcellulose has a viscosity, 2% in water, of about 1,000 cP to about 8,000 cP.

23. The composition of claim 19 wherein the hydroxypropylmethylcellulose has about 15% to about 30% methoxyl substitution and about 5% to about 15% hydroxypropoxyl substitution.

24. The composition of claim 19 wherein the hydroxypropylmethylcellulose has about 15% to about 27% methoxyl substitution and about 7% to about 12% hydroxypropoxyl substitution.

25. A composition of claim 19 that is suitable for providing therapeutically or prophylactically effective inhibition of cyclooxygenase-2 when orally administered to a subject once a day.

26. A composition of claim 19 comprising one or more dose units in a form of discrete solid articles.

27. The composition of claim 26 wherein said articles are tablets.

28. The composition of claim 19 further comprising one or more additional pharmaceutically acceptable excipients selected from lubricants, binding agents, glidants, dyes, fillers and extenders.

29. An orally deliverable pharmaceutical composition comprising a therapeutically effective amount of a selective cyclooxygenase-2 inhibitory drug of low water solubility, a substantial portion or all of the drug being present in beads having a coating comprising a release-extending polymer or copolymer.

30. The composition of claim 29 wherein the polymer or copolymer is selected from hydroxypropylmethylcellulose, hydroxypropylcellulose, hydroxyethylcellulose, ethylcellulose, cellulose acetate and polymers and copolymers of acrylic acid, methacrylic acid and esters thereof.

31. The composition of claim 29 wherein the coating comprises ethylcellulose.

32. The composition of claim 29 wherein the coating comprises a polymer or copolymer of acrylic acid, methacrylic acid and esters thereof.

33. The composition of claim 29 wherein the coating comprises ethylcellulose, hydroxypropylmethylcellulose and a plasticizer.

34. A method of treating a medical condition or disorder in a subject where treatment with a cyclooxygenase-2 inhibitory drug is indicated, comprising orally administering to the subject a composition of claim 1 once a day.

35. The method of claim 34 wherein the condition or disorder is rheumatoid arthritis.

36. The method of claim 34 wherein the condition or disorder is osteoarthritis.

37. The method of claim 34 wherein the condition or disorder, or a symptom of the condition or disorder, is pain.
